(19) United States
(12) Patent Application Publication
(10) Pub. No.: US 2022/0093277 A1
(43) Pub. Date: Mar. 24, 2022

(54) PATH ANALYTICS OF DISEASE VECTORS IN A PHYSICAL SPACE USING SMART FLOOR TILES

(71) Applicant: SCANALYTICS, INC., Milwaukee, WI (US)

(72) Inventor: Joseph Scanlin, Milwaukee, WI (US)

(73) Assignee: SCANALYTICS, INC., Milwaukee, WI (US)

(21) Appl. No.: 17/544,602

(22) Filed: Dec. 7, 2021

Related U.S. Application Data
(63) Continuation-in-part of application No. 17/116,582, filed on Dec. 9, 2020.
Application No. 17/116,582 is a continuation-in-part of application No. 16/696,802, filed on Nov. 26, 2019, now Pat. No. 10,954,677.
(60) Provisional application No. 63/122,644, filed on Dec. 8, 2020.
(60) Provisional application No. 62/956,532, filed on Jan. 2, 2020.

(51) Int. Cl. G16H 50/80

(52) U.S. Cl. CPC G16H 50/80; G16H 10/60

(57) ABSTRACT

A method for tracking potential disease spread in a physical space is disclosed. The method includes receiving, at a first time in the time series from a device in the physical space, first data pertaining to a first initiation event of a first path of a first living creature in the physical space; receiving, at a second time in the time series from smart floor tiles in the physical space, second data pertaining to a first time and location event caused by the first living creature in the physical space, wherein the first time and location event comprises a first initial location of the first living creature in the physical space; and correlating, the first initiation event and the first initial location to generate a first starting point comprising a first starting time and first starting location of a first path of the first living creature in the physical space.

### CROSS-REFERENCES TO RELATED APPLICATIONS

The present application claims priority to and the benefit of U.S. Provisional Patent Application No. 63/122,644, titled “PATH ANALYTICS OF DISEASE VECTORS IN A PHYSICAL SPACE USING SMART FLOOR TILES” filed Dec. 8, 2020, and the present application is a continuation-in-part of U.S. Non-Provisional application Ser. No. 17/116,582, titled “PATH ANALYTICS OF PEOPLE IN A PHYSICAL SPACE USING SMART FLOOR TILES” filed Dec. 9, 2020, which claims priority to U.S. Provisional Application No. 62/956,532, titled “PREVENTION OF FALL EVENTS USING INTERVENTIONS BASED ON DATA ANALYTICS” filed Jan. 2, 2020, and which is a continuation-in-part of U.S. Non-Provisional application Ser. No. 16/696,802, titled “CONNECTED MOULDING FOR USE IN SMART BUILDING CONTROL” filed Nov. 26, 2019, the content of these applications are incorporated herein by reference in their entirety for all purposes.

### TECHNICAL FIELD

This disclosure relates to data analytics. More specifically, this disclosure relates to path analytics of people in a physical space using smart floor tiles.

### BACKGROUND

Certain diseases (e.g., COVID-19) may be more likely to spread to other people by proximity. Certain locations (e.g., hospitals, nursing homes, convention centers, hotels, etc.) provide substantial opportunities for the transmission of contagious diseases like COVID-19. In the case of COVID-19, people have been advised to “socially distance” by staying more than six feet away from other people. COVID-19 may be detectable through indicators, such as by having an elevated temperature. It is considered desirable that those infected with COVID-19 or believed to be infected with COVID-19 be quarantined. Certain entities are engaging in contact tracing, wherein, once a person has been diagnosed with COVID-19, that person is asked who they have been in contact with within a certain time period, to assist in assessing the risk of transmission to others and determining whether others should be tested for COVID-19 or quarantined for some time period.

### SUMMARY

In one embodiment, a method for tracking potential disease spread in a physical space is disclosed. The method includes receiving, at a first time in the time series from a device in the physical space, first data pertaining to a first initiation event of a first path of a first living creature in the physical space. The method further includes receiving, at a second time in the time series from one or more smart floor tiles in the physical space, second data pertaining to a first time and location event caused by the first living creature in the physical space, wherein the first time and location event comprises a first initial location of the first living creature in the physical space. The method further includes correlating, via a processing device, the first initiation event and the first initial location to generate a first starting point comprising a first starting time and first starting location of a first path of the first living creature in the physical space.

In one embodiment, a tangible, non-transitory computer-readable medium stores instructions that, when executed, cause a processing device to perform any operation of any method disclosed herein.

In one embodiment, a system includes a memory device storing instructions and a processing device communicatively coupled to the memory device. The processing device executes the instructions to perform any operation of any method disclosed herein.

Other technical features may be readily apparent to one skilled in the art from the following figures, descriptions, and claims.

### BRIEF DESCRIPTION OF THE DRAWINGS

For a detailed description of example embodiments, reference will now be made to the accompanying drawings in which:

FIGS. 1A-1E illustrate various example configurations of components of a system according to certain embodiments of this disclosure;

FIG. 2 illustrates an example component diagram of a moulding section according to certain embodiments of this disclosure;

FIG. 3 illustrates an example backside view of a moulding section according to certain embodiments of this disclosure;

FIG. 4 illustrates a network and processing context for smart building control according to certain embodiments of this disclosure;

FIG. 5 illustrates aspects of a smart floor tile according to certain embodiments of this disclosure;

FIG. 6 illustrates a master control device according to certain embodiments of this disclosure;

FIG. 7A illustrate an example of a method for generating a path of a person in a physical space using smart floor tiles according to certain embodiments of this disclosure;

FIG. 7B illustrates an example of a method continued from FIG. 7A according to certain embodiments of this disclosure;

FIG. 8 illustrates an example of a method for filtering paths of objects presented on a display screen according to certain embodiments of this disclosure;

FIG. 9 illustrates an example of a method for presenting a longest path of an object in a physical space according to certain embodiments of this disclosure;

FIG. 10 illustrates an example of a method for presenting amount of times objects spent at certain zones in a physical space according to certain embodiments of this disclosure;

FIG. 11 illustrates an example of a method for determining where to place objects based on paths of people according to certain embodiments of this disclosure;

FIG. 12 illustrates an example of a method for overlaying paths of objects based on criteria according to certain embodiments of this disclosure;

FIG. 13A illustrates an example user interface presenting paths of people in a physical space according to certain embodiments of this disclosure;

FIG. 13B illustrates an example user interface presenting a filtered path of a person in a physical space according to certain embodiments of this disclosure;

FIG. 13C illustrates an example user interface presenting information pertaining to paths of people in a physical space according to certain embodiments of this disclosure;

FIG. 13D illustrates an example user interface presenting other information pertaining to a path of a person in a physical space and a recommendation where to place an object in the physical space based on path analytics according to certain embodiments of this disclosure;

FIG. 14 illustrates an example computer system according to embodiments of this disclosure;

FIG. 15A illustrates an example of a method for generating a path of a person in a physical space using smart floor tiles according to certain embodiments of this disclosure; and

FIG. 15B illustrates an example of a method continued from FIG. 15A according to certain embodiments of this disclosure.

### NOTATION AND NOMENCLATURE

Various terms are used to refer to particular system components. Different entities may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

Various terms are used to refer to particular system components. Different entities may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

The terminology used herein is for the purpose of describing particular example embodiments only, and is not intended to be limiting. As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The terms first, second, third, etc. may be used herein to describe various elements, components, regions, layers and/or sections; however, these elements, components, regions, layers and/or sections should not be limited by these terms. These terms may be only used to distinguish one element, component, region, layer or section from another region, layer or section. Terms such as “first,” “second,” and other numerical terms, when used herein, do not imply a sequence or order unless clearly indicated by the context. Thus, a first element, component, region, layer or section discussed below could be termed a second element, component, region, layer or section without departing from the teachings of the example embodiments. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

Spatially relative terms, such as “inner,” “outer,” “beneath,” “below,” “lower,” “above,” “upper,” “top,” “bottom,” and the like, may be used herein. These spatially relative terms can be used for ease of description to describe one element's or feature's relationship to another element(s) or feature(s) as illustrated in the figures. The spatially relative terms may also be intended to encompass different orientations of the device in use, or operation, in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptions used herein interpreted accordingly.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), solid state drives (SSDs), flash memory, or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for other certain words and phrases are provided throughout this patent document. Those of ordinary skill in the art should understand that in many if not most instances, such definitions apply to prior as well as future uses of such defined words and phrases.

The term “moulding” may be spelled as “molding” herein.

### DETAILED DESCRIPTION

The following discussion is directed to various embodiments of the disclosed subject matter. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

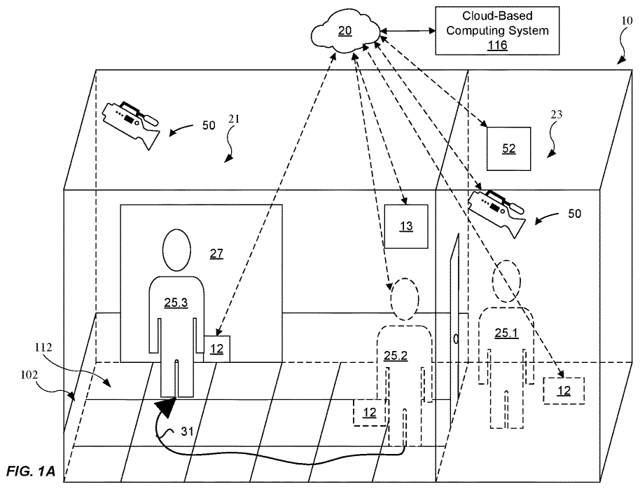

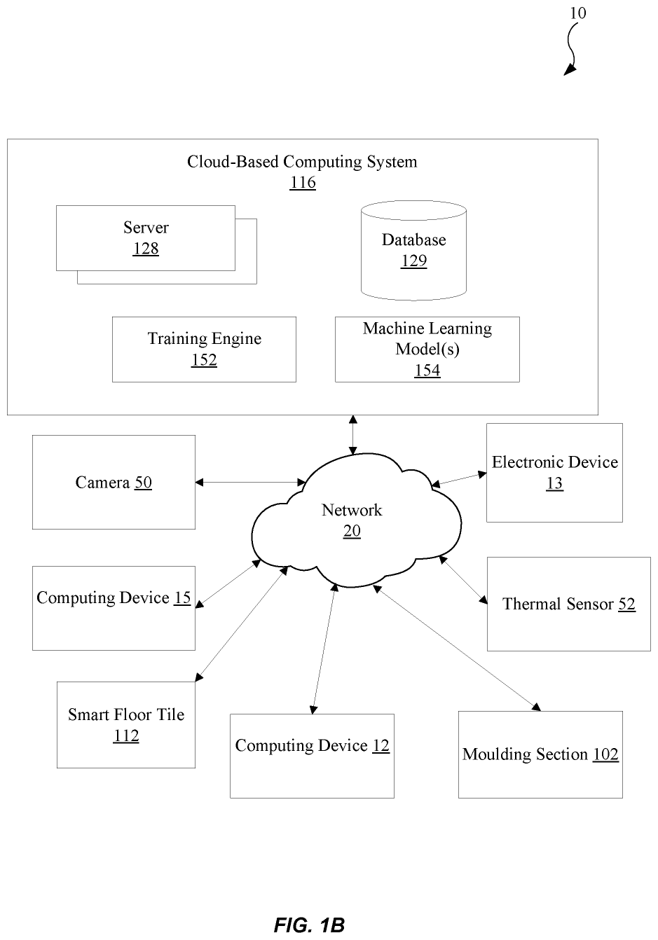

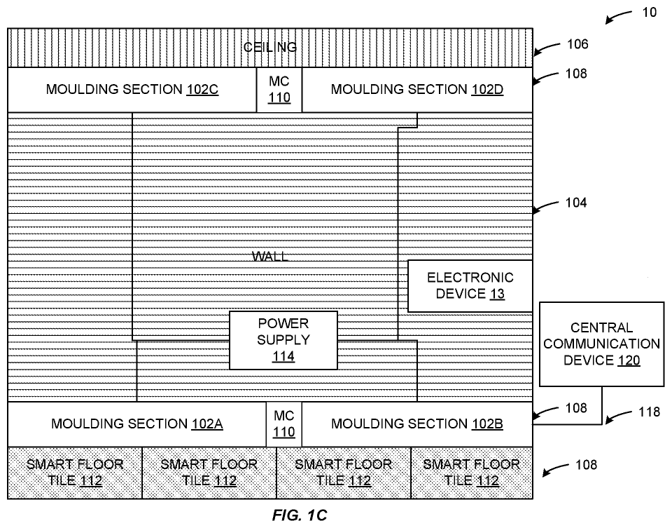

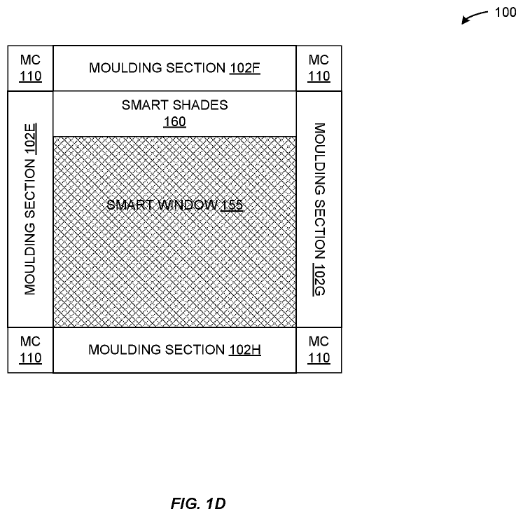

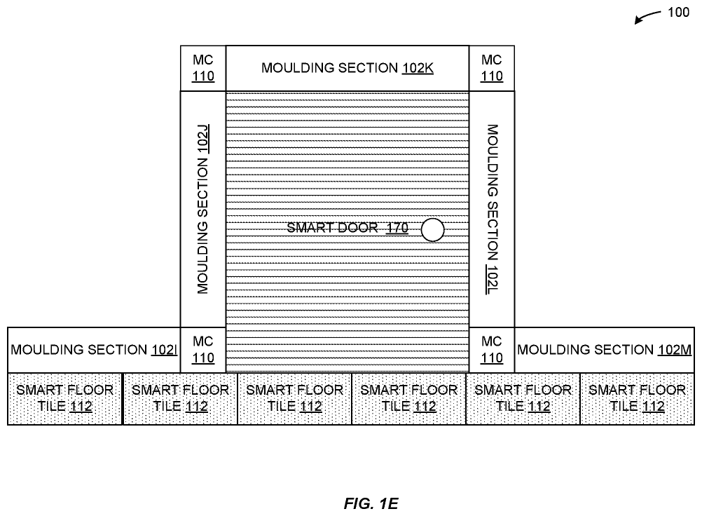

FIGS. 1A through 14, discussed below, and the various embodiments used to describe the principles of this disclosure in this patent document are by way of illustration only and should not be construed in any way to limit the scope of the disclosure.

Embodiments as disclosed herein relate to path analytics for objects in a physical space. For example, the physical space may be a hospital, nursing home, convention center, hotel, or any suitable physical space where people move (e.g., walk, use a wheel chair or motorized cart, etc.) around in a path. Certain locations may be more prone to foot traffic and/or more likely for people to attend due to their proximity to certain other objects (e.g., lobbies, bathrooms, food courts, entrances, exits, etc.). In some instances, certain locations may be more likely for people to attend based on the layout of the physical space and/or the way other locations are arranged in the physical space.

It may be desirable to engage in contact tracing of diseases and disease symptoms at certain locations. For example, it may be beneficial to determine the paths of people that have been or may in the future be determined to have been infected with an infectious disease. It may be desirable to determine the paths of the people in the physical space to better understand which locations are at a higher risk for transmission of diseases. It may be desirable to understand the amounts of time that certain people sped in certain locations or talking to certain people in order to determine the risk of transmission in an interaction. The path analytics may enable determining where to locate certain services in order to reduce risk of transmission of infectious diseases. For example, it may be desirable to separate particularly popular vendors in food courts to spread out the crowds. It may also be desirable to understand where people tend to gather without following social distancing guidelines in order to direct security or supervisory personnel to break up groups or enforce social distancing guidelines. To that end, it may be beneficial to determine the paths of people and which locations in a physical space are more likely to be attended to enable contact tracing or recommend solutions or actions to take in order to reduce the probability of transmission of infectious diseases.

To enable path analytics, some embodiments of the present disclosure may utilize smart floor tiles that are disposed in a physical space where people may move around. For example, the smart floor tiles may be installed in a floor of a convention hall where vendors display objects at booths in certain zones, in a hospital, or in a nursing home. The smart floor tiles may be capable of measuring data (e.g., pressure) associated with footsteps of the people and transmitting the measured data to a cloud-based computing system that analyzes the measured data. In some embodiments, moulding sections, a thermal sensor, and/or a camera may be used to measure the data and/or supplement the data measured by the smart floor tiles. The accuracy of the measurements pertaining to the path of the people may be improved using the smart floor tiles as they measure the physical pressure of the footsteps of the person to track the path of the person and/or other gait characteristics (e.g., width of feet, speed of gait, amount of time spent at certain locations, etc.).

Further, the paths of the people may be correlated with other information, such as job titles of the people, age of the people, gender of the people, employers of the people, detected temperatures of the people, observed labored breathing, and the like. This information may be retrieved from a third party data source and/or data source internal to the cloud-based computing system (e.g., a thermal camera or sensor). For example, the cloud-based computing system may be communicatively coupled with one or more web services (e.g., application programming interfaces) that provide the information to the cloud-based computing system.

The paths that are generated for the people may be overlaid on a virtual representation of the physical space including and/or excluding graphics representing the zones, booths located in the zones, and/or objects displayed in the booths in the physical space. All of the paths of all of the people that move around the physical space during an event, for example, may be overlaid on each other on a user interface presented on a computing device. In some embodiments, a user may select to filter the paths that are presented to just paths of people having a certain job title, to a longest path, to paths that indicate the people visited certain booths, to paths that spent a certain amount of time at a particular zone and/or booth, and the like. The filtering may be performed using any suitable criteria. Accordingly, the disclosed techniques may improve the user's experience using a computing device because an improved user interface that presents desired paths may be provided to the user such that path analytics are enhanced.

The enhanced path analytics may enable the user to make a better determination regarding the layout of facilities. Further, in some embodiments, the cloud-based computing system may analyze the paths and provide contact tracing of people or other living creatures (e.g., a cat or dog, both of which could be potential disease vectors in the physical space. For example, if a person has an elevated temperature, then the cloud-based computing system may recommend that certain other people that person has been in contact with be tested or quarantined.

Barring unforeseeable changes in human locomotion, humans can be expected to generate measurable interactions with buildings through their footsteps on buildings' floors. In some embodiments the smart floor tiles may help realize the potential of a “smart building” by providing, amongst other things, control inputs for a building's environmental control systems using directional occupancy sensing based on occupants' interaction with building surfaces, including, without limitation, floors, interaction with a physical space including their location relative to moulding sections, and climate and airflow systems. Such environmental control systems could act to isolate at risk individuals to reduce the probability of transmission (i.e., by reducing stagnant air around at-risk persons or by placing at-risk persons in isolated air circuits).

The moulding sections, may include a crown moulding, a baseboard, a shoe moulding, a door casing, and/or a window casing, that are located around a perimeter of a physical space. The moulding sections may be modular in nature in that the moulding sections may be various different sizes and the moulding sections may be connected with moulding connectors. The moulding connectors may be configured to maintain conductivity between the connected moulding sections. To that end, each moulding section may include various components, such as electrical conductors, sensors, processors, memories, network interfaces, and so forth that enable communicating data, distributing power, obtaining moulding section sensor data, and so forth. The moulding sections may use various sensors to obtain moulding section sensor data including the location of objects in a physical space as the objects move around the physical space. The moulding sections may use moulding section sensor data to determine a path of the object in the physical space and/or to control other electronic devices (e.g., smart shades, smart windows, smart doors, HVAC system, smart lights, and so forth) in the smart building. Accordingly, the moulding sections may be in wired and/or wireless communication with the other electronic devices. Further, the moulding sections may be in electrical communication with a power supply. The moulding sections may be powered by the power supply and may distribute power to smart floor tiles that may also be in electrical communication with the moulding sections.

A camera may provide a livestream of video data and/or image data to the cloud-based computing system. The camera may be a thermal camera capable of detecting temperatures of objects. The data from the camera may be used to identify certain people in a room and/or track the path of the people in the room. The data from the camera may be used to determine probability of a person being infected (e.g., elevated body temperature) with an infectious disease (e.g., COVID-19). Further, the data may be used to monitor one or more parameters pertaining to a gait of the person to aid in the path analytics. For example, facial recognition may be performed using the data from the camera to identify a person when they first enter a physical space and correlate the identity of the person with the person's path when the person begins to walk on the smart floor tiles.

The cloud-based computing system may monitor one or more parameters of the person based on the measured data from the smart floor tiles, the moulding sections, and/or the camera. The one or more parameters may be associated with the gait of the person and/or the path of the person. Based on the one or more parameters, the cloud-based computing system may determine paths of people in the physical space. The cloud-based computing system may perform any suitable analysis of the paths of the people.

In addition, a technical problem may include determining, from a distal location, when people are in contact with each other and/or within a certain proximity to each other in a physical space. This technical problem is exacerbated if the people in the physical space are not carrying a mobile device that is capable of providing location services. Even when the people are carrying mobile devices, the quality of a signal (e.g., wireless or cellular) may be poor, which may lead to faulty or inaccurate determinations of whether the people come within a certain proximity to each other.

Accordingly, in some embodiments, the present disclosure may provide a technical solution by enabling accurately determining (e.g., via a distal location using a server) when people are in contact with each other and/or within a certain proximity to each other in a physical space. To enable such accurate determination, some embodiments include using measured data from the smart floor tiles, the moulding sections, and/or the camera. Further, thermal data obtained from a thermal sensor in the physical space may determine a temperature of each of the people in the physical space to determine if they exhibit a symptom of a particular disease. The thermal data may be used alone or in conjunction with the measured data to perform a preventative action.

Turning now to the figures, FIGS. 1A-1E illustrate various example configurations of components of a system 10 according to certain embodiments of this disclosure. FIG. 1A visually depicts components of the system in a first room 21 and a second room 23 and FIG. 1B depicts a high-level component diagram of the system 10. For purposes of clarity, FIGS. 1A and 1B are discussed together below.

The first room 21, in this example, is a building that a person 25 is visiting. The first room 21 may be any suitable room that includes a floor capable of being equipped with smart floor tiles 112, moulding sections 102, a camera 50, and/or a thermal sensor 52. The second room 23, in this example, is a entry station or lobby.

When the person initially arrives at the building, the person 25.1 may check in and/or register for entry to the first room 21. As depicted, the person may carry a computing device 12, which may be a smartphone, a laptop, a tablet, a pager, a card, or any suitable computing device. The person 25.1 may use the computing device 12 to check in to the building. For example, the person may 25.1 may swipe the computing device 12 or place it next to a reader that extracts data and sends the data to the cloud-based computing system 116. The data may include an identity of the person 25.1. The reception of the data at the cloud-based computing system 116 may be referred to as an initiation event of a path of an object (e.g., person 25.1) in the physical space (e.g., first room 21) at a first time in a time series. In some embodiments, a camera 50 may send data to the cloud-based computing system 116 that performs facial recognition techniques to determine the identity of the person 25.1. In some embodiments, the thermal sensor 50 may send data to the cloud-based computing system 116 that performs temperature checks against a reference temperature value to determine the probability that the person 25.1 may be infected. Receiving the data from the camera 50 and/or the thermal sensor 52 may also be referred to as an initiation event herein.

Subsequently to the initiation event occurring, the cloud-based computing system 116 may receive data from a first smart floor tile 112 that the person 25.2 steps on at a second time (subsequent to the first time in the time series). The data from the first smart floor tile 112 may occur at a location event that includes an initial location of the person in the physical space. The cloud-based computing device may correlate the initiation event and the initial location to generate a starting point of a path of the person 25.2 in the first room 21.

The person 25.3 may walk around the first room 21 to visit a target location 27. The smart floor tiles 112 may be continuously or continually transmitting measurement data to the cloud-based computing system 116 as the person 25.3 walks from the entrance of the first room 21 to the target location 27. The cloud-based computing system 116 may generate a path 31 of the person 25.3 through the first room 21.

The first room 21 may also include at least one electronic device 13, which may be any suitable electronic device, such as a smart thermostat, smart vacuum, smart light, smart speaker, smart electrical outlet, smart hub, smart appliance, smart television, etc.

Each of the smart floor tiles 112, moulding sections 102, camera 50, thermal sensor 52, computing device 12, and/or electronic device 13 may be capable of communicating, either wirelessly and/or wired, with the cloud-based computing system 116 via a network 20. As used herein, a cloud-based computing system refers, without limitation, to any remote or distal computing system accessed over a network link. Each of the smart floor tiles 112, moulding sections 102, camera 50, computing device 12, and/or electronic device 13 may include one or more processing devices, memory devices, and/or network interface devices.

The network interface devices of the smart floor tiles 112, moulding sections 102, camera 50, thermal sensor 52, computing device 12, and/or electronic device 13 may enable communication via a wireless protocol for transmitting data over short distances, such as Bluetooth, ZigBee, near field communication (NFC), etc. Additionally, the network interface devices may enable communicating data over long distances, and in one example, the smart floor tiles 112, moulding sections 102, camera 50, thermal sensor 52, computing device 12, and/or electronic device 13 may communicate with the network 20. Network 20 may be a public network (e.g., connected to the Internet via wired (Ethernet) or wireless (WiFi)), a private network (e.g., a local area network (LAN), wide area network (WAN), virtual private network (VPN)), or a combination thereof.

The computing device 12 may be any suitable computing device, such as a laptop, tablet, smartphone, or computer. The computing device 12 may include a display that is capable of presenting a user interface. The user interface may be implemented in computer instructions stored on a memory of the computing device 12 and/or computing device 15 and executed by a processing device of the computing device 12. The user interface may be a stand-alone application that is installed on the computing device 12 or may be an application (e.g., website) that executes via a web browser.

The user interface may be generated by the cloud-based computing system 116 and may present various paths of people in the first room 21 on the display screen. The user interface may include various options to filter the paths of the people based on criteria. Also, the user interface may present recommended locations for certain objects in the first room 21. The user interface may be presented on any suitable computing device. For example, computing device 15 may receive and present the user interface to a person interested in the path analytics provided using the disclosed embodiments. The computing device 15 may be any suitable computing device, such as a laptop, tablet, smartphone, or computer.

In some embodiments, the cloud-based computing system 116 may include one or more servers 128 that form a distributed, grid, and/or peer-to-peer (P2P) computing architecture. Each of the servers 128 may include one or more processing devices, memory devices, data storage, and/or network interface devices. The servers 128 may be in communication with one another via any suitable communication protocol. The servers 128 may receive data from the smart floor tiles 112, moulding sections 102, the camera 50, and/or the thermal sensor 52 and monitor a parameter pertaining to a gait of the person 25 based on the data. For example, the data may include pressure measurements obtained by a sensing device in the smart floor tile 112 or temperature of the person 25. The pressure measurements may be used to accurately track footsteps of the person 25, walking paths of the person 25, gait characteristics of the person 25, walking patterns of the person 25 throughout each day, and the like. The servers 128 may determine an amount of gait deterioration based on the parameter. The servers 128 may determine whether a propensity for a fall event for the person 25 satisfies a threshold propensity condition based on (i) the amount of gait deterioration satisfying a threshold deterioration condition, or (ii) the amount of gait deterioration satisfying the threshold deterioration condition within a threshold time period. If the propensity for the fall event for the person 25 satisfies the threshold propensity condition, the servers 128 may select one or more interventions to perform for the person 25 to prevent the fall event from occurring and may perform the one or more selected interventions. The servers 128 may use one or more machine learning models 154 trained to monitor the parameter pertaining to the gait of the person 25 based on the data, determine the amount of gait deterioration based on the parameter, and/or determine whether the propensity for the fall event for the person satisfies the threshold propensity condition.

In some embodiments, the cloud-based computing system 116 may include a training engine 152 and/or the one or more machine learning models 154. The training engine 152 and/or the one or more machine learning models 154 may be communicatively coupled to the servers 128 or may be included in one of the servers 128. In some embodiments, the training engine 152 and/or the machine learning models 154 may be included in the computing device 12, computing device 15, and/or electronic device 13.

The one or more of machine learning models 154 may refer to model artifacts created by the training engine 152 using training data that includes training inputs and corresponding target outputs (correct answers for respective training inputs). The training engine 152 may find patterns in the training data that map the training input to the target output (the answer to be predicted), and provide the machine learning models 154 that capture these patterns. The set of machine learning models 154 may comprise, e.g., a single level of linear or non-linear operations (e.g., a support vector machine [SVM]) or a deep network, i.e., a machine learning model comprising multiple levels of non-linear operations. Examples of such deep networks are neural networks including, without limitation, convolutional neural networks, recurrent neural networks with one or more hidden layers, and/or fully connected neural networks.

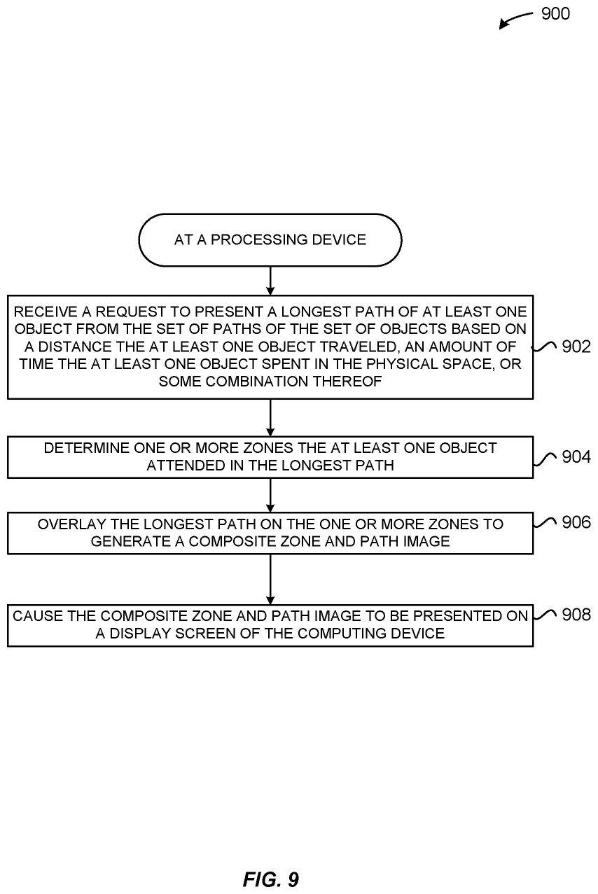

In some embodiments, the training data may include inputs of parameters (e.g., described below with regards to FIG. 9), variations in the parameters, variations in the parameters within a threshold time period, or some combination thereof and correlated outputs of locations of objects to be placed in the first room 21 based on the parameters. That is, in some embodiments, there may be a separate respective machine learning model 154 for each individual parameter that is monitored. The respective machine learning model 154 may output a recommended location for an object based on the parameters (e.g., amount of time people spend at certain locations, paths of people, etc.).

In some embodiments, the cloud-based computing system 116 may include a database 129. The database 129 may store data pertaining to paths of people (e.g., a visual representation of the path, identifiers of the smart floor tiles 112 the person walked on, the amount of time the person stands on each smart floor tile 112 (which may be used to determine an amount of time the person spends at certain booths), and the like), identities of people, recorded temperatures of people, job titles of people, employers of people, age of people, gender of people, residential information of people, and the like. In some embodiments, the database 129 may store data generated by the machine learning models 154, such as recommended locations for objects in the first room 21. Further, the database 129 may store information pertaining to the first room 21, such as the type and location of objects displayed in the first room 21, the booths included in the first room 21, the zones (e.g., boundaries) including the locations the first room (e.g., food courts, bathrooms, etc.) and the like. The database 129 may also store information pertaining to the smart floor tile 112, moulding section 102, the camera 50, and/or the thermal sensor 52, such as device identifiers, addresses, locations, and the like. The database 129 may store paths for people that are correlated with an identity of the person 25. The database 129 may store a map of the first room 21 including the smart floor tiles 112, moulding sections 102, camera 50, any booths 27, and so forth. The database 129 may store video data of the first room 21. The training data used to train the machine learning models 154 may be stored in the database 129.

The camera 50 may be any suitable camera capable of obtaining data including video and/or images and transmitting the video and/or images to the cloud-based computing system 116 via the network 20. The camera 50 may be a thermal (i.e., infrared) camera. The data obtained by the camera 50 may include timestamps for the video and/or images. In some embodiments, the cloud-based computing system 116 may perform computer vision to extract high-dimensional digital data from the data received from the camera 50 and produce numerical or symbolic information. The numerical or symbolic information may represent the parameters monitored pertaining to the path of the person 25 monitored by the cloud-based computing system 116. The video data obtained by the camera 50 may be used for facial recognition of the person 25.

The thermal sensor 52 may be any suitable device (including a thermal camera) capable of detecting temperature information and transmitting the temperature information to the cloud-based computing system 116 via the network 20. The data obtained by the temperature sensor 52 may include timestamps for the video and/or images.

FIGS. 1C-1E depict various example configurations of smart floor tiles 112, and/or moulding sections 102 according to certain embodiments of this disclosure. FIG. 1C depicts an example system 10 that is used in a physical space of a smart building (e.g., care facility). The depicted physical space includes a wall 104, a ceiling 106, and a floor 108 that define a room. Numerous moulding sections 102A, 102B, 102C, and 102D are disposed in the physical space. For example, moulding sections 102A and 102B may form a baseboard or shoe moulding that is secured to the wall 108 and/or the floor 108. Moulding sections 102C and 102D may for a crown moulding that is secured to the wall 108 and/or the ceiling 106. Each moulding section 102A may have different shapes and/or sizes.

The moulding sections 102 may each include various components, such as electrical conductors, sensors, processors, memories, network interfaces, and so forth. The electrical conductors may be partially or wholly enclosed within one or more of the moulding sections. For example, one electrical conductor may be a communication cable that is partially enclosed within the moulding section and exposed externally to the moulding section to electrically couple with another electrical conductor in the wall 108. In some embodiments, the electrical conductor may be communicably connected to at least one smart floor tile 112. In some embodiments, the electrical conductor may be in electrical communication with a power supply 114. In some embodiments, the power supply 114 may provide electrical power that is in the form of mains electricity general-purpose alternating current. In some embodiments, the power supply 114 may be a battery, a generator, or the like.

In some embodiments, the electrical conductor is configured for wired data transmission. To that end, in some embodiments the electrical conductor may be communicably coupled via cable 118 to a central communication device 120 (e.g., a hub, a modem, a router, etc.). Central communication device 120 may create a network, such as a wide area network, a local area network, or the like. Other electronic devices 13 may be in wired and/or wireless communication with the central communication device 120. Accordingly, the moulding section 102 may transmit data to the central communication device 120 to transmit to the electronic devices 13. The data may be control instructions that cause, for example, an the electronic device 13 to change a property. In some embodiments, the moulding section 102A may be in wired and/or wireless communication connection with the electronic device 13 without the use of the central communication device 120 via a network interface and/or cable. The electronic device 13 may be any suitable electronic device capable of changing an operational parameter in response to a control instruction.

In some embodiments, the electrical conductor may include an insulated electrical wiring assembly. In some embodiments, the electrical conductor may include a communications cable assembly. The moulding sections 102 may include a flame-retardant backing layer. The moulding sections 102 may be constructed using one or more materials selected from: wood, vinyl, rubber, fiberboard, metal, plastic, and wood composite materials.

The moulding sections may be connected via one or more moulding connectors 110. A moulding connector 110 may enhance electrical conductivity between two moulding sections 102 by maintaining the conductivity between the electrical conductors of the two moulding sections 102. For example, the moulding connector 110 may include contacts and its own electrical conductor that forms a closed circuit when the two moulding sections are connected with the moulding connector 110. In some embodiments, the moulding connectors 110 may include a fiber optic relay to enhance the transfer of data between the moulding sections 102. It should be appreciated that the moulding sections 102 are modular and may be cut into any desired size to fit the dimensions of a perimeter of a physical space. The various sized portions of the moulding sections 102 may be connected with the moulding connectors 110 to maintain conductivity.

Moulding sections 102 may utilize a variety of sensing technologies, such as proximity sensors, optical sensors, membrane switches, pressure sensors, and/or capacitive sensors, to identify instances of an object proximate or located near the sensors in the moulding sections and to obtain data pertaining to a gait of the person 25. Proximity sensors may emit an electromagnetic field or a beam of electromagnetic radiation (infrared, for instance), and identify changes in the field or return signal. The object being sensed may be any suitable object, such as a human, an animal, a robot, furniture, appliances, and the like. Sensing devices in the moulding section may generate moulding section sensor data indicative of gait characteristics of the person 25, location (presence) of the person 25, the timestamp associated with the location of the person 25, and so forth.

The moulding section sensor data may be used alone or in combination with tile impression data generated by the smart floor tiles 112 and/or image data generated by the camera 50 to perform path analytics for people. For example, the moulding section sensor data may be used to determine a control instruction to generate and to transmit to an electric device 13 and/or the smart floor tile 102A. The control instruction may include changing an operational parameter of the electronic device 13 based on the moulding section sensor data. The control instruction may include instructing the smart floor tile 112 to reset one or more components based on an indication in the moulding section sensor data that the one or more components is malfunctioning and/or producing faulty results. Further, the moulding sections 102 may include a directional indicator (e.g., light) that emits different colors of light, intensities of light, patterns of light, etc. based on path analytics of the cloud-based computing system 116.

In some embodiments, the moulding section sensor data can be used to verify the impression tile data and/or image data of the camera 50 is accurate for generating and analyzing paths of people. Such a technique may improve accuracy of the path analytics. Further, if the moulding section sensor data, the impression tile data, and/or the image data do not align (e.g., the moulding section sensor data does not indicate a path of a person and impression tile data indicates a path of the person), then further analysis may be performed. For example, tests can be performed to determine if there are defective sensors at the corresponding smart floor tile 112 and/or the corresponding moulding section 102 that generated the data. Further, control actions may be performed such as resetting one or more components of the moulding section 102 and/or the smart floor tile 112. In some embodiments, preference to certain data may be made by the cloud-based computing system 116. For example, in one embodiment, preference for the impression tile data may be made over the moulding section sensor data and/or the image data, such that if the impression tile data differs from the moulding section sensor data and/or the image data, the impression tile data is used to perform path analytics.

FIG. 1D illustrates another configuration of the moulding sections 102. In this example, the moulding sections 102E-102H surround a border of a smart window 155. The moulding sections 102 are connected via the moulding connector 110. As may be appreciated, the modular nature of the moulding sections 102 with the moulding connectors 110 enables forming a square around the window. Other shapes may be formed using the moulding sections 102 and the moulding connectors 110.

The moulding sections 102 may be electrically and/or communicably connected to the smart window 155 via electrical conductors and/or interfaces. The moulding sections 102 may provide power to the smart window 155, receive data from the smart window 155, and/or transmit data to the smart window 155. One example smart window includes the ability to change light properties using voltage that may be provided by the moulding sections 102. The moulding sections 102 may provide the voltage to control the amount of light let into a room based on path analytics. For example, if the moulding section sensor data, impression tile data, and/or image data indicates a portion of the first room 21 includes a lot of people, the cloud-based computing system 116 may perform an action by causing the moulding sections 102 to instruct the smart window 155 to change a light property to allow light into the room. In some instances the cloud-based computing system 116 may communicate directly with the smart window 155 (e.g., electronic device 13).

In some embodiments, the moulding sections 102 may use sensors to detect when the smart window 155 is opened. The moulding sections 102 may determine whether the smart window 155 opening is performed at an expected time (e.g., when a home owner is at home) or at an unexpected time (e.g., when the home owner is away from home). The moulding sections 102, the camera 50, and/or the smart floor tile 112 may sense the occupancy patterns of certain objects (e.g., people) in the space in which the moulding sections 102 are disposed to determine a schedule of the objects. The schedule may be referenced when determining if an undesired opening (e.g., break-in event) occurs and the moulding sections 102 may be communicatively to an alarm system to trigger the alarm when the certain event occurs.

The schedule may also be referenced when determining a medical condition of the person 25. For example, if the schedule indicates that the person 25 went to the bathroom a certain number of times (e.g., 10) within a certain time period (e.g., 1 hour), the cloud-based computing system 116 may determine that the person has a urinary tract infection (UTI) and may perform an intervention, such as transmitting a message to the computing device 12 of the person 25. The message may indicate the potential UTI and recommend that the person 25 schedules an appointment with a medical personnel.

As depicted, at least moulding section 102F is electrically and/or communicably coupled to smart shades 160. Again, the cloud-based computing system 116 may cause the moulding section 102F to control the smart shades 160 to extend or retract to control the amount of light let into a room. In some embodiments, the cloud-based computing system 116 may communicate directly with the smart shades 160.

FIG. 1E illustrates another configuration of the moulding sections 102 and smart floor tiles 112. In this example, the moulding sections 102E-102H surround a majority of a border of a smart door 170. The moulding sections 102J, 102K, and 102L and/or the smart floor tile 112 may be electrically and/or communicably connected to the smart door 170 via electrical conductors and/or interfaces. The moulding sections 102 and/or smart floor tiles 112 may provide power to the smart door 170, receive data from the smart door 170, and/or transmit data to the smart door 170. In some embodiments, the moulding sections 102 and/or smart floor tiles 112 may control operation of the smart door 170. For example, if the moulding section sensor data and/or impression tile data indicates that no one is present in a house for a certain period of time, the moulding sections 102 and/or smart floor tiles 112 may determine a locked state of the smart door 170 and generate and transmit a control instruction to the smart door 170 to lock the smart door 170 if the smart door 170 is in an unlocked state.

In another example, the moulding section sensor data, impression tile data, and/or the image data may be used to generate gait profiles for people in a smart building (e.g., care facility). When a certain person is in the room near the smart door 170, the cloud-based computing device 116 may detect that person's presence based on the data received from the smart floor tiles, moulding sections 102, and/or camera 50. In some embodiments, if the person 25 is detected near the smart door 170, the cloud-based computing system 116 may determine whether the person 25 has a particular medical condition (e.g., alzheimers) and/or a flag is set that the person should not be allowed to leave the smart building. If the person is detected near the smart door 170 and the person 25 has the particular medical condition and/or the flag set, then the cloud-based computing system 116 may cause the moulding sections 102 and/or smart floor tiles 112 to control the smart door 170 to lock the smart door 170. In some embodiments, the cloud-based computing system 116 may communicate directly with the smart door 170 to cause the smart door 170 to lock.

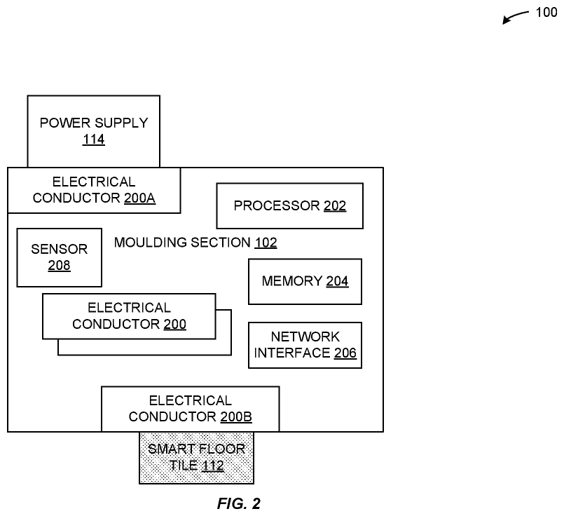

FIG. 2 illustrates an example component diagram of a moulding section 102 according to certain embodiments of this disclosure. As depicted, the moulding section 102 includes numerous electrical conductors 200, a processor 202, a memory 204, a network interface 206, and a sensor 208. More or fewer components may be included in the moulding section 102. The electrical conductors may be insulated electrical wiring assemblies, communications cable assemblies, power supply assemblies, and so forth. As depicted, one electrical conductor 200A may be in electrical communication with the power supply 114, and another electrical conductor 200B may be communicably connected to at least one smart floor tile 112.

In various embodiments, the moulding section 102 further comprises a processor 202. In the non-limiting example shown in FIG. 2, processor 202 is a low-energy microcontroller, such as the ATMEGA328P by Atmel Corporation. According to other embodiments, processor 202 is the processor provided in other processing platforms, such as the processors provided by tablets, notebook or server computers.

In the non-limiting example shown in FIG. 2, the moulding section 102 includes a memory 204. According to certain embodiments, memory 204 is a non-transitory memory containing program code to implement, for example, generation and transmission of control instructions, networking functionality, the algorithms for generating and analyzing locations, presence, paths, and/or tracks, and the algorithms for performing path analytics as described herein.

Additionally, according to certain embodiments, the moulding section 102 includes the network interface 206, which supports communication between the moulding section 102 and other devices in a network context in which smart building control using directional occupancy sensing and path analytics is being implemented according to embodiments of this disclosure. In the non-limiting example shown in FIG. 2, network interface 206 includes circuitry 635 for sending and receiving data using Wi-Fi, including, without limitation at 900 MHz, 2.8 GHz and 5.0 GHz. Additionally, network interface 206 includes circuitry, such as Ethernet circuitry 640 for sending and receiving data (for example, smart floor tile data) over a wired connection. In some embodiments, network interface 206 further comprises circuitry for sending and receiving data using other wired or wireless communication protocols, such as Bluetooth Low Energy or Zigbee circuitry. The network interface 206 may enable communicating with the cloud-based computing device 116 via the network 20.

Additionally, according to certain embodiments, network interface 206 which operates to interconnect the moulding device 102 with one or more networks. Network interface 206 may, depending on embodiments, have a network address expressed as a node ID, a port number or an IP address. According to certain embodiments, network interface 206 is implemented as hardware, such as by a network interface card (NIC). Alternatively, network interface 206 may be implemented as software, such as by an instance of the java.net.NetworkInterface class. Additionally, according to some embodiments, network interface 206 supports communications over multiple protocols, such as TCP/IP as well as wireless protocols, such as 3G or Bluetooth. Network interface 206 may be in communication with the central communication device 120 in FIG. 1.

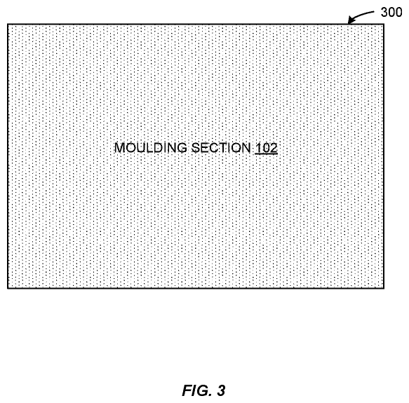

FIG. 3 illustrates an example backside view 300 of a moulding section 102 according to certain embodiments of this disclosure. As depicted by the dots 300, the backside of the moulding section 102 may include a fire-retardant backing layer positioned between the moulding section 102 and the wall to which the moulding section 102 is secured.

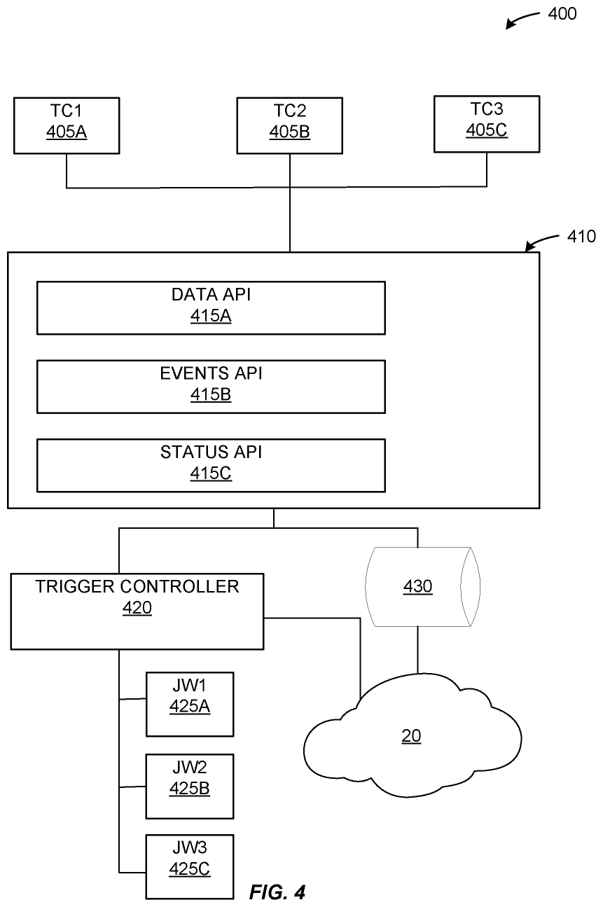

FIG. 4 illustrates a network and processing context 400 for smart building control using directional occupancy sensing and path analytics according to certain embodiments of this disclosure. The embodiment of the network context 400 shown in FIG. 4 is for illustration only and other embodiments could be used without departing from the scope of the present disclosure.

In the non-limiting example shown in FIG. 4, a network context 400 includes one or more tile controllers 405A, 405B and 405C, an API suite 410, a trigger controller 420, job workers 425A-425C, a database 430 and a network 435.

According to certain embodiments, each of tile controllers 405A-405C is connected to a smart floor tile 112 in a physical space. Tile controllers 405A-405C generate floor contact data (also referred to as impression tile data herein) from smart floor tiles in a physical space and transmit the generated floor contact data to API suite 410. In some embodiments, data from tile controllers 405A-405C is provided to API suite 410 as a continuous stream. In the non-limiting example shown in FIG. 4, tile controllers 405A-405C provide the generated floor contact data from the smart floor tile to API suite 410 via the internet. Other embodiments, wherein tile controllers 405A-405C employ other mechanisms, such as a bus or Ethernet connection to provide the generated floor data to API suite 410 are possible and within the intended scope of this disclosure.

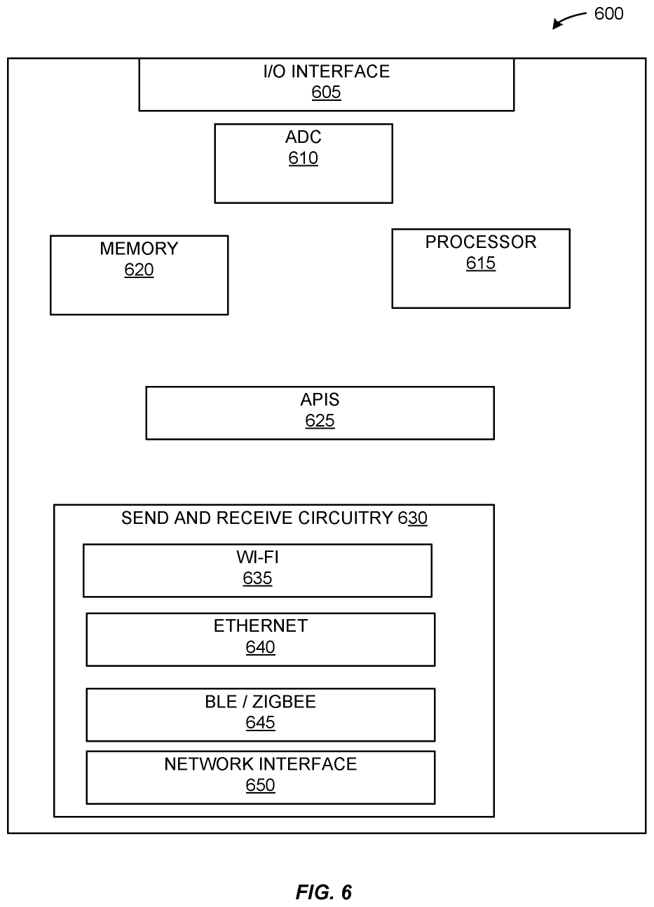

According to some embodiments, API suite 410 is embodied on a server 128 in the cloud-based computing system 116 connected via the internet to each of tile controllers 405A-405C. According to some embodiments, API suite is embodied on a master control device, such as master control device 600 shown in FIG. 6 of this disclosure. In the non-limiting example shown in FIG. 4, API suite 410 comprises a Data Application Programming Interface (API) 415A, an Events API 415B and a Status API 215C.

In some embodiments, Data API 415A is an API for receiving and recording tile data from each of tile controllers 405A-405C. Tile events include, for example, raw, or minimally processed data from the tile controllers, such as the time and data a particular smart floor tile was pressed and the duration of the period during which the smart floor tile was pressed. According to certain embodiments, Data API 415A stores the received tile events in a database such as database 430. In the non-limiting example shown in FIG. 4, some or all of the tile events are received by API suite 410 as a stream of event data from tile controllers 405A-405C, Data API 415A operates in conjunction with trigger controller 420 to generate and pass along triggers breaking the stream of tile event data into discrete portions for further analysis.

According to various embodiments, Events API 415B receives data from tile controllers 405A-405C and generates lower-level records of instantaneous contacts where a sensor of the smart floor tile is pressed and released.

In the non-limiting example shown in FIG. 4, Status API 415C receives data from each of tile controllers 405A-405C and generates records of the operational health (for example, CPU and memory usage, processor temperature, whether all of the sensors from which a tile controller receives inputs is operational) of each of tile controllers 405A-405C. According to certain embodiment, status API 415C stores the generated records of the tile controllers' operational health in database 430.

According to some embodiments, trigger controller 420 operates to orchestrate the processing and analysis of data received from tile controllers 405A-405C. In addition to working with data API 415A to define and set boundaries in the data stream from tile controllers 405A-405C to break the received data stream into tractably sized and logically defined “chunks” for processing, trigger controller 420 also sends triggers to job workers 425A-425C to perform processing and analysis tasks. The triggers comprise identifiers uniquely identifying each data processing job to be assigned to a job worker. In the non-limiting example shown in FIG. 4, the identifiers comprise: 1.) a sensor identifier (or an identifier otherwise uniquely identifying the location of contact); 2.) a time boundary start identifying a time in which the smart floor tile went from an idle state (for example, an completely open circuit, or, in the case of certain resistive sensors, a baseline or quiescent current level) to an active state (a closed circuit, or a current greater than the baseline or quiescent level); and 3.) a time boundary end defining the time in which a smart floor tile returned to the idle state.

In some embodiments, each of job workers 425A-425C corresponds to an instance of a process performed at a computing platform, (for example, cloud-based computing system 116 in FIG. 1) for determining paths and performing an analysis of the paths (e.g., such as filtering paths based on criteria, recommending a location of an object based on the paths, predicting a propensity for a fall event and performing an intervention based on the propensity). Instances of processes may be added or subtracted depending on the number of events or possible events received by API suite 410 as part of the data stream from tile controllers 405A-205C. According to certain embodiments, job workers 425A-425C perform an analysis of the data received from tile controllers 405A-405C, the analysis having, in some embodiments, two stages. A first stage comprises deriving footsteps, and paths, or tracks, from impression tile data. A second stage comprises characterizing those footsteps, and paths, or tracks, to determine gait characteristics of the person 25. The paths and/or gait characteristics may be presented to an online dashboard (in some embodiments, provided by a UI on an electronic device, such as computing device 12 or 15 in FIG. 1) and to generate control signals for devices (e.g., the computing devices 12 and/or 15, the electronic device 15, the moulding sections 102, the camera 50, and/or the smart floor tile 112 in FIG. 1) controlling operational parameters of a physical space where the smart floor impression tile data were recorded.

In the non-limiting example shown in FIG. 4, job workers 425A-425C perform the constituent processes of a method for analyzing smart floor tile impression tile data and/or moulding section sensor data to generate paths, or tracks. In some embodiments, an identity of the person 25 may be correlated with the paths or tracks. For example, if the person scanned an ID badge when entering the physical space, their path may be recorded when the person takes their first step on a smart floor tile and their path may be correlated with an identifier received from scanning the badge. In this way, the paths of various people may be recorded (e.g., in a convention hall). This may be beneficial if certain people have desirable job titles (e.g., chief executive officer (CEO), vice president, president, etc.) and/or work at desirable client entities. For example, in some embodiments, the path of a CEO may be tracked during a convention to determine which booths the CEO stopped at and/or an amount of time the CEO spent at each booth. Such data may be used to determine where to place certain booths in the future. For example, if a booth was visited by a threshold number of people having a certain title for a certain period of time, a recommendation may be generated and presented that recommends relocating the booth to a location in the convention hall that is more easily accessible to foot traffic. Likewise, if it is determined that a booth has poor visitation frequency based on the paths, or tracks, of attendees at the convention, a recommendation may be generated to relocate the booth to another location that is more easily accessible to foot traffic. In some embodiments, the machine learning models 154 may be trained to determine the paths, or tracks, of the people having various job titles and working for desired client entities, analyze their paths (e.g., which location the people visited, how long the people visited those locations, etc.), and generate recommendations.

According to certain embodiments, the method comprises the operations of obtaining impression image data, impression tile data, and/or moulding section sensor data from database 430, cleaning the obtained image data, impression tile data, and/or moulding section sensor data and reconstructing paths using the cleaned data. In some embodiments, cleaning the data includes removing extraneous sensor data, removing gaps between image data, impression tile data, and/or moulding section sensor data caused by sensor noise, removing long image data, impression tile data, and/or moulding section sensor data caused by objects placed on smart floor tiles, by objects placed in front of moulding sections, by objects stationary in image data, by defective sensors, and sorting image data, impression tile data, and/or moulding section sensor data by start time to produce sorted image data, impression tile data, and/or moulding section sensor data. According to certain embodiments, job workers 425A-425C perform processes for reconstructing paths by implementing algorithms that first cluster image data, impression tile data, and/or moulding section sensor data that overlap in time or are spatially adjacent. Next, the clustered data is searched, and pairs of image data, impression tile data, and/or moulding section sensor data that start or end within a few milliseconds of one another are combined into footsteps and/or locations of the object, which are then linked together to form footsteps and/or locations. Footsteps and/or locations are further analyzed and linked to create paths.

According to certain embodiments, database 430 provides a repository of raw and processed image data, smart floor tile impression tile data, and/or moulding section sensor data, as well as data relating to the health and status of each of tile controllers 405A-405C and moulding sections 102. In the non-limiting example shown in FIG. 4, database 430 is embodied on a server machine communicatively connected to the computing platforms providing API suite 410, trigger controller 420, and upon which job workers 425A-425C execute. According to some embodiments, database 430 is embodied on the cloud-based computing system 116 as the database 129.

In the non-limiting example shown in FIG. 4, the computing platforms providing trigger controller 420 and database 430 are communicatively connected to one or more network(s) 20. According to embodiments, network 20 comprises any network suitable for distributing impression tile data, image data, moulding section sensor data, determined paths, determined gait deterioration of a parameter, determine propensity for a fall event, and control signals (e.g., interventions) based on determined propensities for fall events, including, without limitation, the internet or a local network (for example, an intranet) of a smart building.

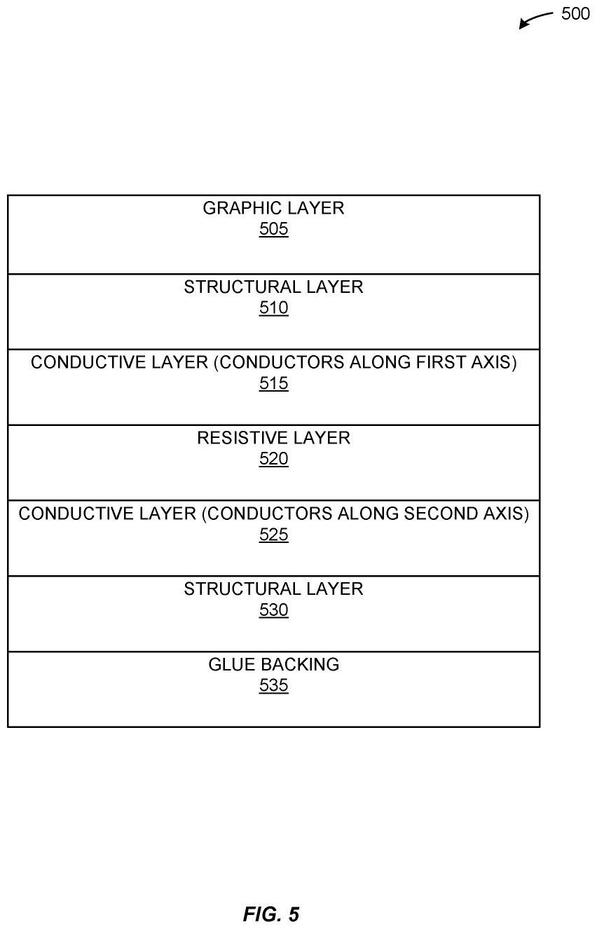

Smart floor tiles utilizing a variety of sensing technologies, such as membrane switches, pressure sensors and capacitive sensors, to identify instances of contact with a floor are within the contemplated scope of this disclosure. FIG. 5 illustrates aspects of a resistive smart floor tile 500 according to certain embodiments of the present disclosure. The embodiment of the resistive smart floor tile 500 shown in FIG. 5 is for illustration only and other embodiments could be used without departing from the scope of the present disclosure.

In the non-limiting example shown in FIG. 5, a cross section showing the layers of a resistive smart floor tile 500 is provided. According to some embodiments, the resistance to the passage of electrical current through the smart floor tile varies in response to contact pressure. From these changes in resistance, values corresponding to the pressure and location of the contact may be determined. In some embodiments, resistive smart floor tile 500 may comprise a modified carpet or vinyl floor tile, and have dimensions of approximately 2′×2′.

According to certain embodiments, resistive smart floor tile 500 is installed directly on a floor, with graphic layer 505 comprising the top-most layer relative to the floor. In some embodiments, graphic layer 505 comprises a layer of artwork applied to smart floor tile 500 prior to installation. Graphic layer 505 can variously be applied by screen printing or as a thermal film.

According to certain embodiments, a first structural layer 510 is disposed, or located, below graphic layer 505 and comprises one or more layers of durable material capable of flexing at least a few thousandths of an inch in response to footsteps or other sources of contact pressure. In some embodiments, first structural layer 510 may be made of carpet, vinyl or laminate material.

According to some embodiments, first conductive layer 515 is disposed, or located, below structural layer 510. According to some embodiments, first conductive layer 515 includes conductive traces or wires oriented along a first axis of a coordinate system. The conductive traces or wires of first conductive layer 515 are, in some embodiments, copper or silver conductive ink wires screen printed onto either first structural layer 510 or resistive layer 520. In other embodiments, the conductive traces or wires of first conductive layer 515 are metal foil tape or conductive thread embedded in structural layer 510. In the non-limiting example shown in FIG. 5, the wires or traces included in first conductive layer 515 are capable of being energized at low voltages on the order of 5 volts. In the non-limiting example shown in FIG. 5, connection points to a first sensor layer of another smart floor tile or to tile controller are provided at the edge of each smart floor tile 500.

In various embodiments, a resistive layer 520 is disposed, or located, below conductive layer 515. Resistive layer 520 comprises a thin layer of resistive material whose resistive properties change under pressure. For example, resistive layer 320 may be formed using a carbon-impregnated polyethylete film.

In the non-limiting example shown in FIG. 5, a second conductive layer 525 is disposed, or located, below resistive layer 520. According to certain embodiments, second conductive layer 525 is constructed similarly to first conductive layer 515, except that the wires or conductive traces of second conductive layer 525 are oriented along a second axis, such that when smart floor tile 500 is viewed from above, there are one or more points of intersection between the wires of first conductive layer 515 and second conductive layer 525. According to some embodiments, pressure applied to smart floor tile 500 completes an electrical circuit between a sensor box (for example, tile controller 425 as shown in FIG. 4) and smart floor tile, allowing a pressure-dependent current to flow through resistive layer 520 at a point of intersection between the wires of first conductive layer 515 and second conductive layer 525. The pressure-dependent current may represent a measurement of pressure and the measurement of pressure may be transmitted to the cloud-based computing system 116.

In some embodiments, a second structural layer 530 resides beneath second conductive layer 525. In the non-limiting example shown in FIG. 5, second structural layer 530 comprises a layer of rubber or a similar material to keep smart floor tile 500 from sliding during installation and to provide a stable substrate to which an adhesive, such as glue backing layer 535 can be applied without interference to the wires of second conductive layer 525.

The foregoing description is purely descriptive and variations thereon are contemplated as being within the intended scope of this disclosure. For example, in some embodiments, smart floor tiles according to this disclosure may omit certain layers, such as glue backing layer 535 and graphic layer 505 described in the non-limiting example shown in FIG. 5.

According to some embodiments, a glue backing layer 535 comprises the bottom-most layer of smart floor tile 500. In the non-limiting example shown in FIG. 5, glue backing layer 535 comprises a film of a floor tile glue.

FIG. 6 illustrates a master control device 600 according to certain embodiments of this disclosure. FIG. 6 illustrates a master control device 600 according to certain embodiments of this disclosure. The embodiment of the master control device 600 shown in FIG. 6 is for illustration only and other embodiments could be used without departing from the scope of the present disclosure.

In the non-limiting example shown in FIG. 6, master control device 600 is embodied on a standalone computing platform connected, via a network, to a series of end devices (e.g., tile controller 405A in FIG. 4) in other embodiments, master control device 600 connects directly to, and receives raw signals from, one or more smart floor tiles (for example, smart floor tile 500 in FIG. 5). In some embodiments, the master control device 600 is implemented on a server 128 of the cloud-based computing system 116 in FIG. 1B and communicates with the smart floor tiles 112, the moulding sections 102, the camera 50, the computing device 12, the computing device 15, and/or the electronic device 13.

According to certain embodiments, master control device 600 includes one or more input/output interfaces (I/O) 605. In the non-limiting example shown in FIG. 6, I/O interface 605 provides terminals that connect to each of the various conductive traces of the smart floor tiles deployed in a physical space. Further, in systems where membrane switches or smart floor tiles are used as mat presence sensors, I/O interface 605 electrifies certain traces (for example, the traces contained in a first conductive layer, such as conductive layer 515 in FIG. 5) and provides a ground or reference value for certain other traces (for example, the traces contained in a second conductive layer, such as conductive layer 525 in FIG. 5). Additionally, I/O interface 605 also measures current flows or voltage drops associated with occupant presence events, such as a person's foot squashing a membrane switch to complete a circuit, or compressing a resistive smart floor tile, causing a change in a current flow across certain traces. In some embodiments, I/O interface 605 amplifies or performs an analog cleanup (such as high or low pass filtering) of the raw signals from the smart floor tiles in the physical space in preparation for further processing.

In some embodiments, master control device 600 includes an analog-to-digital converter (“ADC”) 610. In embodiments where the smart floor tiles in the physical space output an analog signal (such as in the case of resistive smart floor tile), ADC 610 digitizes the analog signals. Further, in some embodiments, ADC 610 augments the converted signal with metadata identifying, for example, the trace(s) from which the converted signal was received, and time data associated with the signal. In this way, the various signals from smart floor tiles can be associated with touch events occurring in a coordinate system for the physical space at defined times. While in the non-limiting example shown in FIG. 6, ADC 610 is shown as a separate component of master control device 600, the present disclosure is not so limiting, and embodiments wherein ADC 610 is part of, for example, I/O interface 605 or processor 615 are contemplated as being within the scope of this disclosure.

In various embodiments, master control device 600 further comprises a processor 615. In the non-limiting example shown in FIG. 6, processor 615 is a low-energy microcontroller, such as the ATMEGA328P by Atmel Corporation. According to other embodiments, processor 615 is the processor provided in other processing platforms, such as the processors provided by tablets, notebook or server computers.

In the non-limiting example shown in FIG. 6, master control device 600 includes a memory 620. According to certain embodiments, memory 620 is a non-transitory memory containing program code to implement, for example, APIs 625, networking functionality and the algorithms for generating and analyzing paths described herein.

Additionally, according to certain embodiments, master control device 600 includes one or more Application Programming Interfaces (APIs) 625. In the non-limiting example shown in FIG. 6, APIs 625 include APIs for determining and assigning break points in one or more streams of smart floor tile data and/or moulding section sensor data and defining data sets for further processing. Additionally, in the non-limiting example shown in FIG. 6, APIs 625 include APIs for interfacing with a job scheduler (for example, trigger controller 420 in FIG. 4) for assigning batches of data to processes for analysis and determination of paths. According to some embodiments, APIs 625 include APIs for interfacing with one or more reporting or control applications provided on a client device. Still further, in some embodiments, APIs 625 include APIs for storing and retrieving image data, smart floor tile data, and/or moulding section sensor data in one or more remote data stores (for example, database 430 in FIG. 4, database 129 in FIG. 1B, etc.).

According to some embodiments, master control device 600 includes send and receive circuitry 630, which supports communication between master control device 600 and other devices in a network context in which smart building control using directional occupancy sensing is being implemented according to embodiments of this disclosure. In the non-limiting example shown in FIG. 6, send and receive circuitry 630 includes circuitry 635 for sending and receiving data using Wi-Fi, including, without limitation at 900 MHz, 2.8 GHz and 5.0 GHz. Additionally, send and receive circuitry 630 includes circuitry, such as Ethernet circuitry 640 for sending and receiving data (for example, smart floor tile data) over a wired connection. In some embodiments, send and receive circuitry 630 further comprises circuitry for sending and receiving data using other wired or wireless communication protocols, such as Bluetooth Low Energy or Zigbee circuitry.

Additionally, according to certain embodiments, send and receive circuitry 630 includes a network interface 650, which operates to interconnect master control device 600 with one or more networks. Network interface 650 may, depending on embodiments, have a network address expressed as a node ID, a port number or an IP address. According to certain embodiments, network interface 650 is implemented as hardware, such as by a network interface card (NIC). Alternatively, network interface 650 may be implemented as software, such as by an instance of the java.net.NetworkInterface class. Additionally, according to some embodiments, network interface 650 supports communications over multiple protocols, such as TCP/IP as well as wireless protocols, such as 3G or Bluetooth.

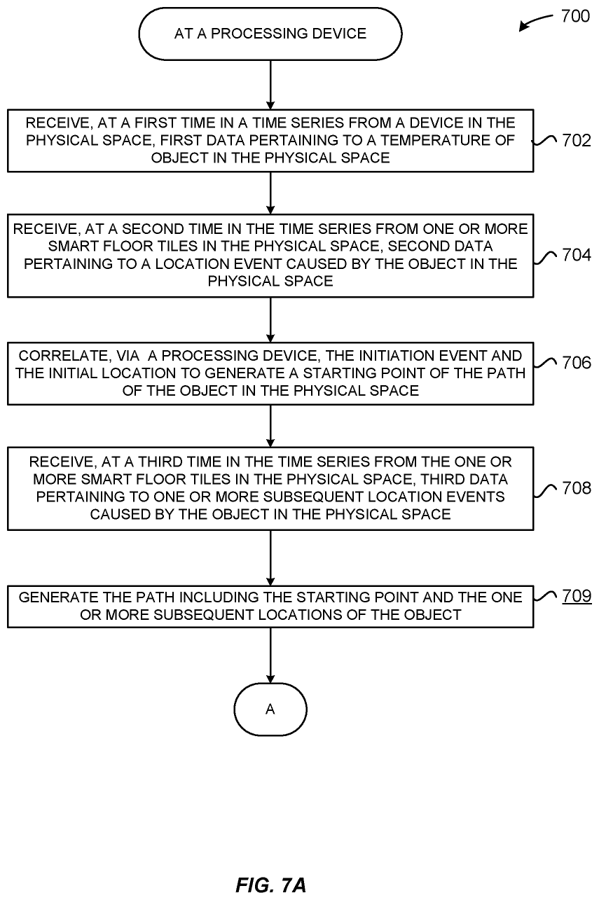

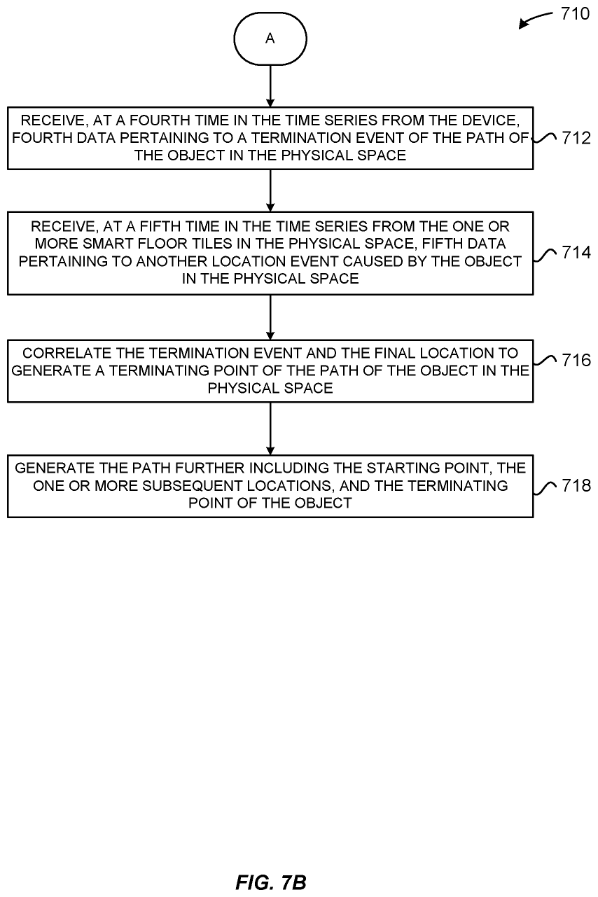

FIG. 7A illustrate an example of a method 700 for generating a path of a person in a physical space using smart floor tiles 112 according to certain embodiments of this disclosure. The method 700 may be performed by processing logic that may include hardware (circuitry, dedicated logic, etc.), software, or a combination of both. The method 700 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 700. The method 700 may be implemented as computer instructions stored on a memory device and executable by the one or more processors. In certain implementations, the method 700 may be performed by a single processing thread. Alternatively, the method 700 may be performed by two or more processing threads, each thread implementing one or more individual functions, routines, subroutines, or operations of the methods.

At block 702, the processing device may receive, at a first time in a time series, from a device (e.g., camera 50, reader device, etc.) in a physical space (first room 21), first data pertaining to an initiation event of the path of the object (e.g., person 25) in the physical space. The first data may include an identity of the person, employment position of the person in an entity, a job title of the person, an entity identity that employs the person, a gender of the person, an age of the person, a timestamp of the data, a temperature of the person, and the like. The initiation event may correspond to the person checking in for an event being held in the physical space. In some embodiments, when the device is a camera 50, the processing device may perform facial recognition techniques using facial image data received from the camera 50 to determine an identity of the person. The processing device may obtain information pertaining to the person based on the identity of the person. The information may include an entity for which the person works, an employment position of the person within the entity, or some combination thereof.

At block 704, the processing device may receive, at a second time in the time series from one or more smart floor tiles 112 in the physical space, second data pertaining to a location event caused by the object in the physical space. The location event may include an initial location of the object in the physical space. The initial location may be generated by one or more detected forces at the one or more smart floor tiles 112. The second data may be impression tile data received when the person steps onto a first smart floor tile 112 in the physical space. In some embodiments, the person may be standing on the first smart floor tile 112 when the initiation event occurs. That is, the initiation event and the location event may occur contemporaneously at substantially the same time in the time series. In some embodiments, the first time and the second time may differ less than a threshold period of time, or the first time and the second time may be substantially the same. The location event may include data pertaining to the one or more smart tiles 112 the object pressed, such as an identifier of the one or more smart floor tiles 112, a timestamp of when the one or more smart floor tiles 112 changed from an idle state to an active state, a duration of being in the active state, and the like.

At block 706, the processing device may correlate the initiation event and the initial location to generate a starting point of a path of the object in the physical space. In some embodiments, the starting point may be overlaid on a virtual representation of the physical space and the path of the object may be generated and presented in real-time or near real-time as the object moves around the physical space.

At block 708, the processing device may receive, at a third time in the time series from the one or more smart floor tiles 112 in the physical space, third data pertaining to one or more subsequent location events caused by the object in the physical space. The one or more subsequent location events may include one or more subsequent locations of the object in the physical space. The one or more subsequent location events may include data pertaining to the one or more smart tiles 112 the object pressed, such as an identifier of the one or more smart floor tiles 112, a timestamp of when the one or more smart floor tiles 112 changed from an idle state to an active state, a duration of being in the active state, and the like.

At block 709, the processing device may generate the path of the object including the starting point and the one or more subsequent locations of the object.

FIG. 7B illustrates an example of a method 710 continued from FIG. 7A according to certain embodiments of this disclosure. The method 710 may be performed by processing logic that may include hardware (circuitry, dedicated logic, etc.), software, or a combination of both. The method 710 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 710. The method 710 may be implemented as computer instructions stored on a memory device and executable by the one or more processors. In certain implementations, the method 710 may be performed by a single processing thread. Alternatively, the method 710 may be performed by two or more processing threads, each thread implementing one or more individual functions, routines, subroutines, or operations of the methods.

At block 712, the processing device may receive, at a fourth time in the time series from a device (e.g., camera 50, reader, etc.), fourth data pertaining to a termination event of the path of the object in the physical space.

At block 714, the processing device may receive, at a fifth time in the time series from the one or more smart floor tiles 112 in the physical space, fifth data pertaining to another location event caused by the object in the physical space. The another location event may correspond to when the user leaves the physical space (e.g., by checking out with a badge or any electronic device). The another location event may include a final location of the object in the physical space. The another location event may include data pertaining to the one or more smart tiles 112 the object pressed, such as an identifier of the one or more smart floor tiles 112, a timestamp of when the one or more smart floor tiles 112 changed from an idle state to an active state, a duration of being in the active state, and the like.

At block 716, the processing device may correlate the termination event and the final location to generate a terminating point of the path of the object in the physical space.

At block 718, the processing device may generate the path using the starting point, the one or more subsequent locations, and the terminating point of the object. Block 718 may result in the full path of the object in the physical space. The full path may be presented on a user interface of a computing device.

In some embodiments, the processing device may generate a second path for a second person in the physical space. The processing device may generate an overlay image by overlaying the path of the first person with the second path of the second object in a virtual representation of the physical space. The different paths may be represented using different or the same visual elements (e.g., color, boldness, etc.). The processing device may cause the overlay image to be presented on a computing device.

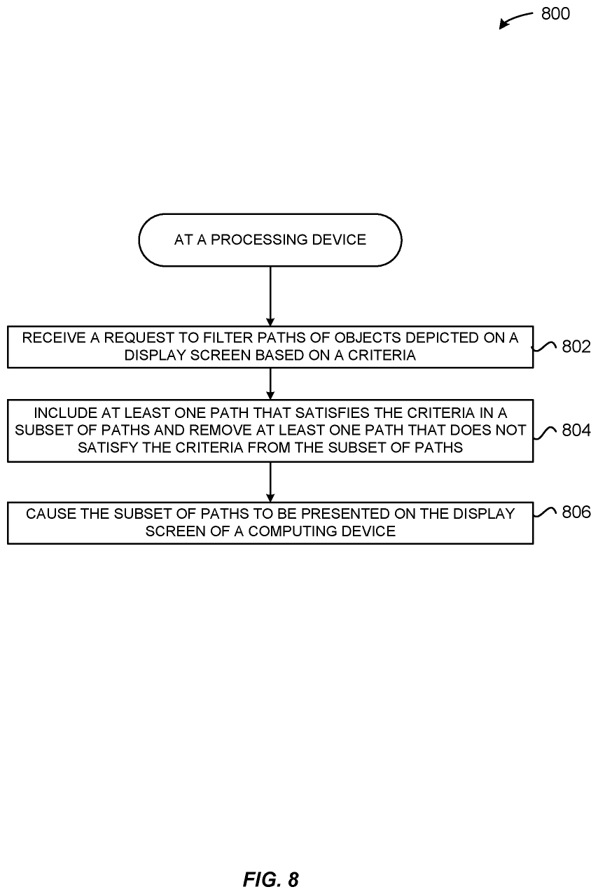

FIG. 8 illustrates an example of a method 800 for filtering paths of objects presented on a display screen according to certain embodiments of this disclosure. The method 800 may be performed by processing logic that may include hardware (circuitry, dedicated logic, etc.), software, or a combination of both. The method 800 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 800. The method 800 may be implemented as computer instructions stored on a memory device and executable by the one or more processors. In certain implementations, the method 800 may be performed by a single processing thread. Alternatively, the method 800 may be performed by two or more processing threads, each thread implementing one or more individual functions, routines, subroutines, or operations of the methods.

At block 802, the processing device may receive a request to filter paths of objects depicted on a user interface of a display screen based on a criteria. The criteria may be employment position, job title, entity identity for which people work, gender, age, or some combination thereof.

At block 804, the processing device may include at least one path that satisfies the criteria in a subset of paths and remove at least one path that does not satisfy the criteria from the subset of paths. For example, if the user selects to view paths of people having a manager position, the processing device may include the paths of all manager positions and remove other paths of people that do not have the manager position.

At block 806, the processing device may cause the subset of paths to be presented on the display screen of a computing device. The subset of paths may provide an improved user interface that increases the user's experience using the computing device because it includes only the desired paths of people in the physical area. Further, computing resources may be reduced by generating the subset of paths because fewer paths may be generated based on the criteria. Also less data may be transmitted over the network to the computing device displaying the subset because there are fewer paths in the subset based on the criteria.

FIG. 9 illustrates an example of a method 900 for presenting a longest path of an object in a physical space according to certain embodiments of this disclosure. The method 900 may be performed by processing logic that may include hardware (circuitry, dedicated logic, etc.), software, or a combination of both. The method 900 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 900. The method 900 may be implemented as computer instructions stored on a memory device and executable by the one or more processors. In certain implementations, the method 900 may be performed by a single processing thread. Alternatively, the method 900 may be performed by two or more processing threads, each thread implementing one or more individual functions, routines, subroutines, or operations of the methods.

At block 902, the processing device may receive a request to present a longest path of at least one object from the set of paths of the set of objects (e.g., people) based on a distance at least one object traveled, an amount of time the at least one object spent in the physical space, or some combination thereof.

At block 904, the processing device may determine one or more zones the at least one object attended in the longest path. The one or more zones may be determined using a virtual representation of the physical space and selecting the zones including smart floor tiles 112 through which the path of the at least one object traversed.

At block 906, the processing device may overlay the longest path of the at least one object on the one or more zones to generate a composite zone and path image.

At block 908, the processing device may cause the composite zone and path image to be presented on a display screen of the computing device. In some embodiments, the shortest path may also be selected and presented on the display screen. The longest path and the shortest path may be presented concurrently. In some embodiments, any suitable length of path in any combination may be selected and presented on a virtual representation of the physical space as desired.

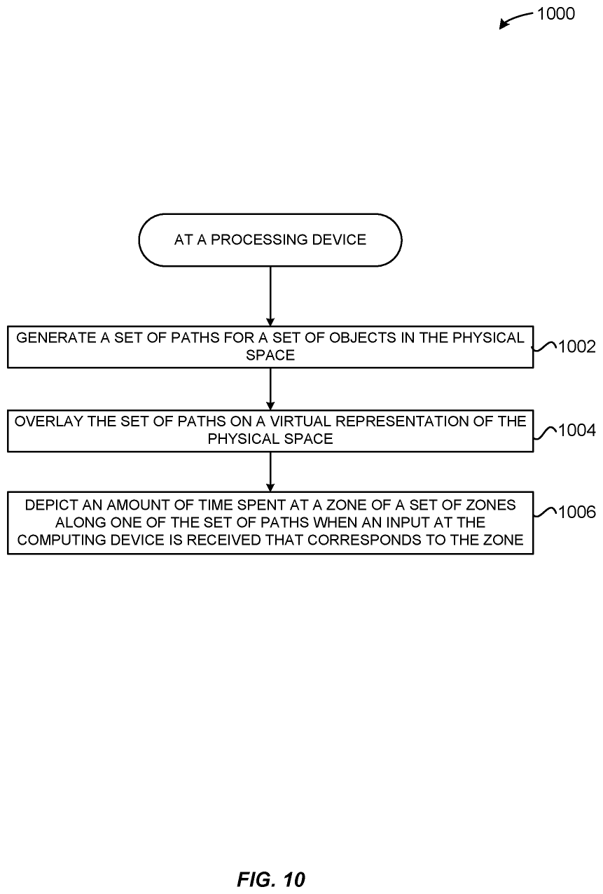

FIG. 10 illustrates an example of a method 1000 for presenting amount of times objects spent at certain zones in a physical space according to certain embodiments of this disclosure. The method 1000 may be performed by processing logic that may include hardware (circuitry, dedicated logic, etc.), software, or a combination of both. The method 1000 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 1000. The method 1000 may be implemented as computer instructions stored on a memory device and executable by the one or more processors. In certain implementations, the method 1000 may be performed by a single processing thread. Alternatively, the method 1000 may be performed by two or more processing threads, each thread implementing one or more individual functions, routines, subroutines, or operations of the methods.

At block 1002, the processing device may generate a set of paths for a set of objects in the physical space. At block 1004, the processing device may overlay the set of paths on a virtual representation of the physical space.

At block 1006, the processing device may depict an amount of time spent at a zone of a set of zones along one of the set of paths when an input at the computing device is received that corresponds to the zone. In some embodiments, the user may select any point on the path of any person to determine the amount of time that person spent at a location at the selected point. Granular location and duration details may be provided using the data obtained via the smart floor tiles 112.

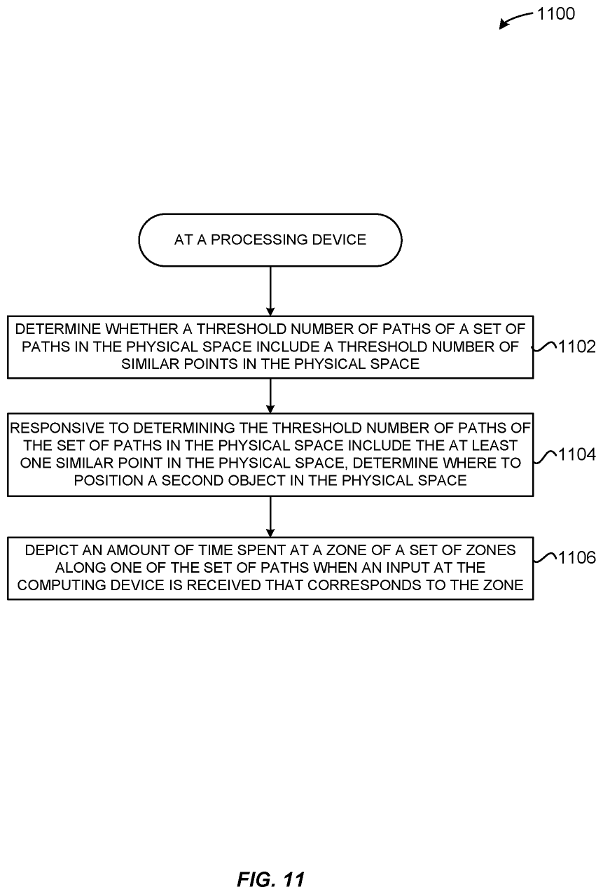

FIG. 11 illustrates an example of a method 1100 for determining where to place objects based on paths of people according to certain embodiments of this disclosure. The method 1100 may be performed by processing logic that may include hardware (circuitry, dedicated logic, etc.), software, or a combination of both. The method 1100 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 1100. The method 1100 may be implemented as computer instructions stored on a memory device and executable by the one or more processors. In certain implementations, the method 1100 may be performed by a single processing thread. Alternatively, the method 1100 may be performed by two or more processing threads, each thread implementing one or more individual functions, routines, subroutines, or operations of the methods.

At block 1102, the processing device may determine whether a threshold number of paths of a set of paths in the physical space include a threshold number of similar points in the physical space. At block 1104, responsive to determining the threshold number of paths of the set of paths in the physical space include the at least one similar point in the physical space, the processing device may determine where to position a second object in the physical space. At block 1106, the processing device may depict an amount of time spent at a zone of a set of zones along one of the set of paths when an input at the computing device is received that corresponds to the zone, a person, a path, a booth, or the like.

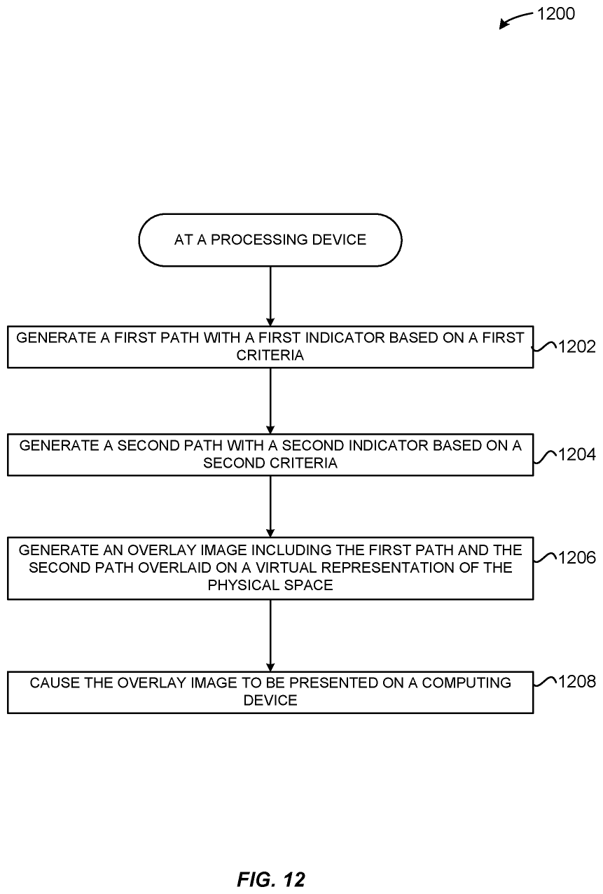

FIG. 12 illustrates an example of a method 1200 for overlaying paths of objects based on criteria according to certain embodiments of this disclosure. The method 1200 may be performed by processing logic that may include hardware (circuitry, dedicated logic, etc.), software, or a combination of both. The method 1200 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 1200. The method 1200 may be implemented as computer instructions stored on a memory device and executable by the one or more processors. In certain implementations, the method 1200 may be performed by a single processing thread. Alternatively, the method 1200 may be performed by two or more processing threads, each thread implementing one or more individual functions, routines, subroutines, or operations of the methods.

At block 1202, the processing device may generate a first path with a first indicator based on a first criteria. The criteria may be job title, company name, age, gender, longest path, shortest path, etc. The first indicator may be a first color for the first path.

At block 1204, the processing device may generate a second path with a second indicator based on a second criteria. At block 1206, the processing device may generate an overlay image including the first path and the second path overlaid on a virtual representation of the physical space. At block 1208, the processing device may cause the overlay image to be presented on a computing device.

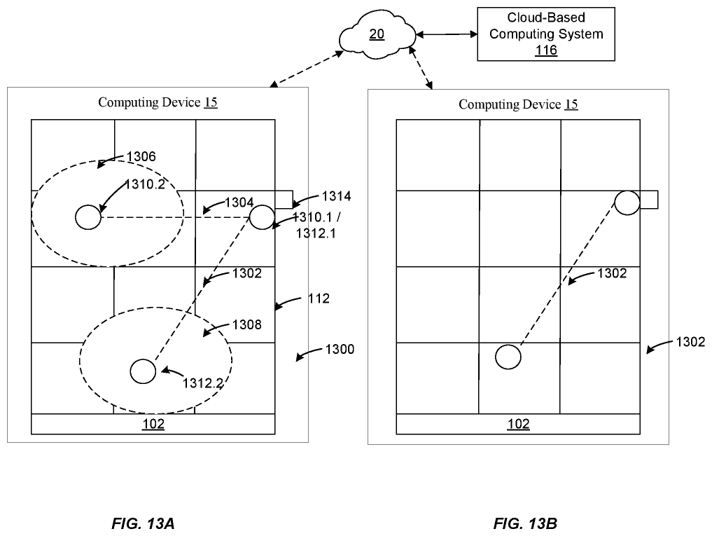

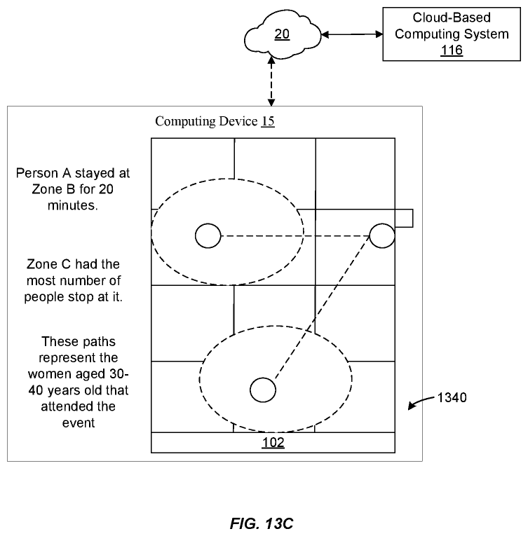

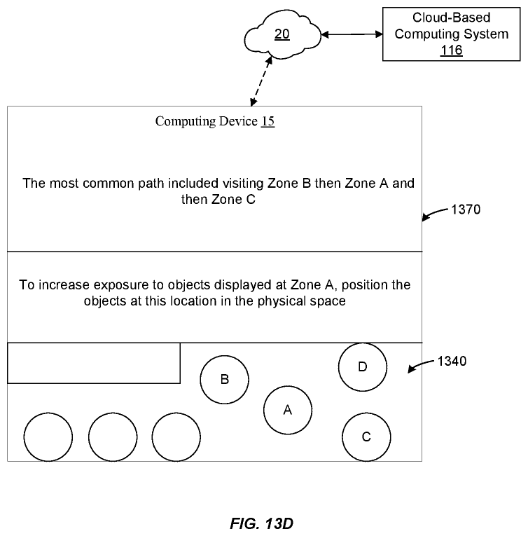

FIG. 13A illustrates an example user interface 1300 presenting paths 1300 and 1304 of people in a physical space according to certain embodiments of this disclosure. More particularly, the user interface 1300 presents a virtual representation of the first room 21, for example, from an above perspective. The user interface 1300 presents the smart floor tiles 112 and/or moulding section 102 that are arranged in the physical space. The user interface 1300 may include a visual representation mapping various zones 1306 and 1308 including various booths in the physical space.

An entrance to the physical space may include a device 1314 at which the user checks in for the event being held in the physical space. The device 1314 may be a reader device and/or a camera 50. The device 1314 may send data to the cloud-based computing system 116 to perform the methods disclosed herein.

For example, the data may be included in an initiation event that is used to generate a starting point of the path of the person. When the person enters the physical space, the person may press one or more first smart floor tiles 112 that transmit measurement data to the cloud-based computing system 116. The measurement data may be included in a location event and may include an initial location of the person in the physical space. The initial location and the initiation event may be used to generate the starting position of the path of the person. The measurement data obtained by the smart floor tiles 112 and sent to the cloud-based computing system 116 may be used during later location events and a termination location event to generate a full path of the person.

As depicted, two starting points 1310.1 and 1312.1 are overlaid on a smart floor tile 112 in the user interface 1300. Starting point 1310.1 is included as part of path 1304 and starting point 1312.1 is included as part of path 1302. Termination points 1310.2 and 1312.2. The termination point 1310.2 ends in zone 1306 and termination point 1312.2 ends in zone 1308. If the user places the cursor or selects any portion of the path (e.g., using a touchscreen), additional details of the paths 1304 and 1302 may be presented. For example, a duration of time the person spent at any of the points in the paths 1304 may be presented.

FIG. 13B illustrates an example user interface 1302 presenting a filtered path of a person in a physical space according to certain embodiments of this disclosure. In some embodiments, the paths presented in the user interface 1302 may be filtered based on any suitable criteria. For example, the user may select to view the paths of a person having a certain employment positon (e.g., a chief level position), and the user interface 1300 presents the path 1302 of the person having the certain employment position and removes the path 1304 of the person that does not have that employment position.

FIG. 13C illustrates an example user interface 1304 presenting information pertaining to paths of people in a physical space according to certain embodiments of this disclosure. As depicted, the user interface 1340 presents “Person A stayed at Zone B for 20 minutes”, “Zone C had the most number of people stop at it”, and “These paths represent the women aged 30-40 years old that attended the event.” As may be appreciated, the improve user interface 1304 may greatly enhance the experience of a user using the computing device 15 as the analytics enabled and disclosed herein may be very beneficial. Any suitable subset of paths may be generated using any suitable criteria.

FIG. 13D illustrates an example user interface 1370 presenting other information pertaining to a path of a person in a physical space and a recommendation where to place an object in the physical space based on path analytics according to certain embodiments of this disclosure. As depicted, the user interface 1370 presents “The most common path included visiting Zone B then Zone A and then Zone C”. The cloud-based computing system 116 may analyze the paths by comparing them to determine the most common path, the least common path, the durations spent at each zone, booth, or object in the physical space, and the like.

The user interface 1370 also presents “To increase exposure to objects displayed at Zone A, position the objects at this location in the physical space”. A visual representation 1372 presents the recommended location for objects in Zone A relative to other Zones B, C, and D. Accordingly, the cloud-based computing system 116 may determine the ideal locations for increasing traffic and/or attendance in zones and may recommend where to locate the zones, the booths in the zones, and/or the objects displayed at particular booths based on path analytics performed herein.

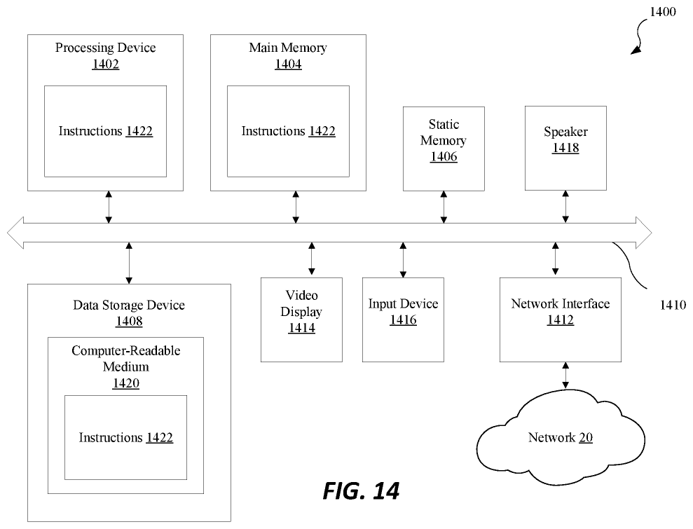

FIG. 14 illustrates an example computer system 1400, which can perform any one or more of the methods described herein. In one example, computer system 1400 may include one or more components that correspond to the computing device 12, the computing device 15, one or more servers 128 of the cloud-based computing system 116, the electronic device 13, the camera 50, the moulding section 102, the smart floor tile 112, or one or more training engines 152 of the cloud-based computing system 116 of FIG. 1B. The computer system 1400 may be connected (e.g., networked) to other computer systems in a LAN, an intranet, an extranet, or the Internet. The computer system 1400 may operate in the capacity of a server in a client-server network environment. The computer system 1400 may be a personal computer (PC), a tablet computer, a laptop, a wearable (e.g., wristband), a set-top box (STB), a personal Digital Assistant (PDA), a smartphone, a camera, a video camera, or any device capable of executing a set of instructions (sequential or otherwise) that specify actions to be taken by that device. Some or all of the components computer system 1400 may be included in the camera 50, the moulding section 102, and/or the smart floor tile 112. Further, while only a single computer system is illustrated, the term “computer” shall also be taken to include any collection of computers that individually or jointly execute a set (or multiple sets) of instructions to perform any one or more of the methods discussed herein.

The computer system 1400 includes a processing device 1402, a main memory 1404 (e.g., read-only memory (ROM), solid state drive (SSD), flash memory, dynamic random access memory (DRAM) such as synchronous DRAM (SDRAM)), a static memory 1406 (e.g., solid state drive (SSD), flash memory, static random access memory (SRAM)), and a data storage device 1408, which communicate with each other via a bus 1410.

Processing device 1402 represents one or more general-purpose processing devices such as a microprocessor, central processing unit, or the like. More particularly, the processing device 1402 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1402 may also be one or more special-purpose processing devices such as an application specific integrated circuit (ASIC), a field programmable gate array (FPGA), a digital signal processor (DSP), network processor, or the like. The processing device 1402 is configured to execute instructions for performing any of the operations and steps discussed herein.

The computer system 1400 may further include a network interface device 1412. The computer system 1400 also may include a video display 1414 (e.g., a liquid crystal display (LCD) or a cathode ray tube (CRT)), one or more input devices 1416 (e.g., a keyboard and/or a mouse), and one or more speakers 1418 (e.g., a speaker). In one illustrative example, the video display 1414 and the input device(s) 1416 may be combined into a single component or device (e.g., an LCD touch screen).

The data storage device 1416 may include a computer-readable medium 1420 on which the instructions 1422 embodying any one or more of the methodologies or functions described herein are stored. The instructions 1422 may also reside, completely or at least partially, within the main memory 1404 and/or within the processing device 1402 during execution thereof by the computer system 1400. As such, the main memory 1404 and the processing device 1402 also constitute computer-readable media. The instructions 1422 may further be transmitted or received over a network via the network interface device 1412.

While the computer-readable storage medium 1420 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

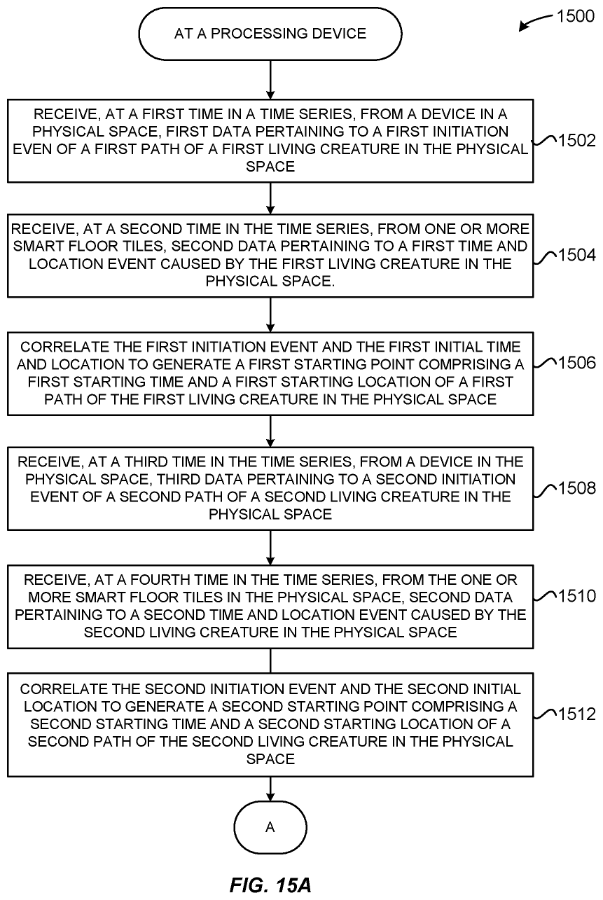

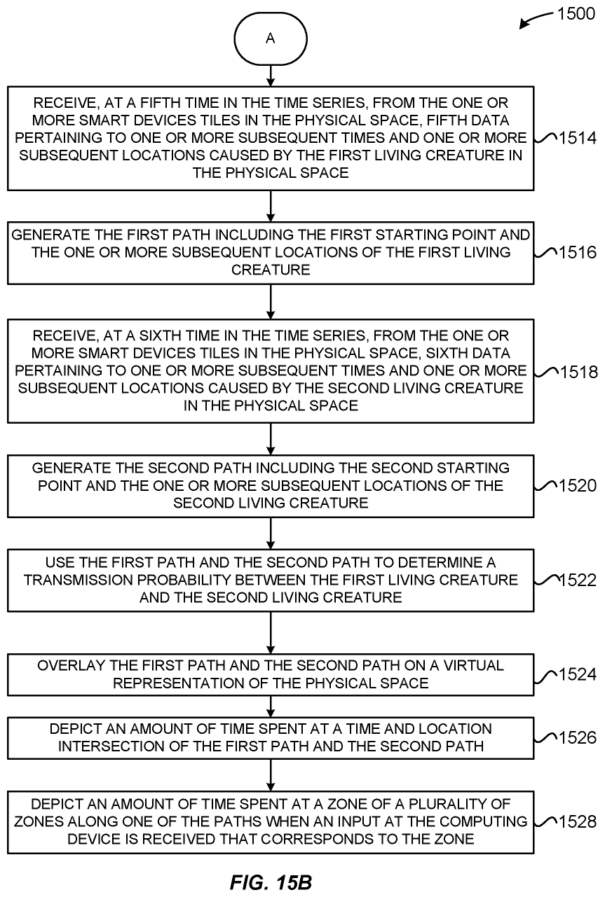

FIG. 15 illustrate an example of a method 1500 for tracking potential disease spread between living creatures within a physical space using smart floor tiles 112 according to certain embodiments of this disclosure. The method 700 may be performed by processing logic that may include hardware (circuitry, dedicated logic, etc.), software, or a combination of both. The method 700 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 700. The method 700 may be implemented as computer instructions stored on a memory device and executable by the one or more processors. In certain implementations, the method 700 may be performed by a single processing thread. Alternatively, the method 700 may be performed by two or more processing threads, each thread implementing one or more individual functions, routines, subroutines, or operations of the methods.

At block 1502, the processing device may receive, at a first time in the time series, from a device in the physical space (e.g., camera 50, reader device, thermal sensor 52, etc.), first data pertaining to a first initiation event of a first path of a first living creature (e.g., person 25) in the physical space. The first data may include a gender of the person, an age of the person, a disease risk factor of the person, whether the person is wearing a face mask, an identity of the person, an employment position of the person in an entity, the entity for which the person works, a timestamp of the data, and the like. The first initiation event may correspond to the person checking in to the physical space (i.e., signing in at the lobby). In some embodiments, when the device is a camera 50, the processing device may perform facial recognition techniques using facial image data received from the camera 50 to determine an identity of the person. In some embodiments, when the device is a thermal sensor 52, the processing device may compare a detected temperature of the person to a threshold value above which the person is considered to have an elevated likelihood of being infected by an infectious disease (e.g., COVID-19). The processing device may obtain information pertaining to the person based on the identity of the person. The information may include an entity for which the person works, an employment position of the person within the entity, a medical history of the person, or some combination thereof.

At block 1504, the processing device may receive, at a second time in the time series from one or more smart floor tiles (e.g., smart floor tiles 122) in the physical space, second data pertaining to a first time and location event caused by the first living creature in the physical space, wherein the first time and location event comprises a first initial location of the first living creature in the physical space. The first time and location event may include an initial location of the person in the physical space. The initial location may be generated by one or more detected forces at the one or more smart floor tiles 112. The second data may be impression tile data received when the person steps onto a first smart floor tile 112 in the physical space. In some embodiments, the person may be standing on the first smart floor tile 112 when the initiation event occurs. That is, the initiation event and the time and location event may occur contemporaneously at substantially the same time in the time series. In some embodiments, the first time and the second time may differ less than a threshold period of time, or the first time and the second time may be substantially the same. The time and location event may include data pertaining to the one or more smart tiles 112 the person pressed, such as an identifier of the one or more smart floor tiles 112, a timestamp of when the one or more smart floor tiles 112 changed from an idle state to an active state, a duration of being in the active state, and the like.

At block 1506, the processing device may correlate, the first initiation event and the first initial time and location to generate a first starting point comprising a first starting time and first starting location of a first path of the first living creature in the physical space. In some embodiments, the starting point may be overlaid on a virtual representation of the physical space and the path of the object may be generated and presented in real-time or near real-time as the object moves around the physical space.

At block 1508, the processing device may receive, at a third time in the time series, from a device in the physical space (e.g., smart floor tiles 112, moulding sections 102, camera 50, reader device, thermal sensor 52, etc.), third data pertaining to a second initiation event of a second path of a second living creature (e.g., another person 25) in the physical space. The third data may include a gender of the person, an age of the person, a disease risk factor of the person, whether the person is wearing a face mask, an identity of the person, an employment position of the person in an entity, the entity for which the person works, a timestamp of the data, and the like. The second initiation event may correspond to the person checking in to the physical space (i.e., signing in at the lobby). In some embodiments, when the device is a camera 50, the processing device may perform facial recognition techniques using facial image data received from the camera 50 to determine an identity of the person. In some embodiments, when the device is a thermal sensor 52, the processing device may compare a detected temperature of the person to a threshold value above which the person is considered to have an elevated likelihood of being infected by an infectious disease (e.g., COVID-19). The processing device may obtain information pertaining to the person based on the identity of the person. The information may include an entity for which the person works, an employment position of the person within the entity, a medical history of the person, or some combination thereof.

At block 1510, the processing device may receive, at a fourth time in the time series from one or more smart floor tiles (e.g., smart floor tiles 112) in the physical space, second data pertaining to a second time and location event caused by the second living creature in the physical space, wherein the second time and location event comprises a second initial location of the second living creature in the physical space. The second time and location event may include an initial location of the second living creature in the physical space. The initial location may be generated by one or more detected forces at the one or more smart floor tiles 112. The second data may be impression tile data received when the second person steps onto a first smart floor tile 112 in the physical space. In some embodiments, the second person may be standing on the first smart floor tile 112 when the initiation event occurs. That is, the initiation event and the time and location event may occur contemporaneously at substantially the same time in the time series. In some embodiments, the first time and the second time may differ less than a threshold period of time, or the first time and the second time may be substantially the same. The time and location event may include data pertaining to the one or more smart tiles 112 the person pressed, such as an identifier of the one or more smart floor tiles 112, a timestamp of when the one or more smart floor tiles 112 changed from an idle state to an active state, a duration of being in the active state, and the like.

At block 1512, the processing device may correlate the second initiation event and the second initial location to generate a second starting point comprising a second starting time and a second starting location of a first path of the second living creature in the physical space. In some embodiments, the starting point may be overlaid on a virtual representation of the physical space and the path of the second living creature may be generated and presented in real-time or near real-time as the second living creature moves around the physical space.

At block 1514, the processing device may receive, at a fifth time in the time series from the one or more smart devices tiles in the physical space, fifth data pertaining to one or more first subsequent time and location events caused by the first living creature in the physical space. The one or more first subsequent time and location events include one or more first subsequent times and one or more first subsequent locations of the first living creature in the physical space. The times and locations may be generated by one or more detected forces at the one or more smart floor tiles 112. The fifth data may be impression tile data received when the person steps onto another smart floor tile 112 in the physical space. The time and location event may include data pertaining to the one or more smart tiles 112 the person pressed, such as an identifier of the one or more smart floor tiles 112, a timestamp of when the one or more smart floor tiles 112 changed from an idle state to an active state, a duration of being in the active state, and the like.

At block 1516, the processing device may generate the first path including the starting point and the one or more subsequent locations of the first living creature.

At block 1518, the processing device may receive, at a sixth time in the time series from the one or more smart devices tiles in the physical space, sixth data pertaining to one or more second subsequent time and location events caused by the second living creature in the physical space. The one or more second subsequent time and location events include one or more second subsequent times and one or more second subsequent locations of the second living creature in the physical space. The times and locations may be generated by one or more detected forces at the one or more smart floor tiles 112. The sixth data may be impression tile data received when the second person steps onto another smart floor tile 112 in the physical space. The time and location event may include data pertaining to the one or more smart tiles 112 the second person pressed, such as an identifier of the one or more smart floor tiles 112, a timestamp of when the one or more smart floor tiles 112 changed from an idle state to an active state, a duration of being in the active state, and the like.

At block 1520, the processing device may generate the second path including the second starting point and the one or more subsequent locations of the second living creature.

At block 1522, the processing device may use the first path and the second path to determine a transmission probability between the first living creature and the second living creature. The transmission probability is the probability that, if the first living creature had a transmissible disease, the first living creature passed on that transmissible disease to the second living creature. For example, the processing device can calculate the transmission probability using how close the first living creature got to the second living creature (i.e., the distance between the first creature and the second creature, whether social distancing regulations or recommendations were followed), how much time the first living creature spent in proximity to the second living creature, whether the first living creature was wearing personal protective equipment (e.g., a mask), whether the second creature was wearing personal protective equipment. The transmission probability may be based solely on the closest distance between the first living creature and the second living creature. The transmission probability may be compared to a threshold transmission probability (i.e., a set probability that may correspond to desired actions to be taken, such as required testing or quarantining). Further, in some embodiments, the transmission probability may be based on the detected temperature of each of the first and second living creature.

If the transmission probability for a living creature is above a threshold amount, then a preventative action may be performed by the cloud-based computing system 116. The preventative action may include causing a user device 12 of the living creature to perform a function. That is, the cloud-based computing system 116 may distally control the user device 12 of the person in a physical space separate from where the server is located. The function performed by the user device 12 may include presenting a notification indicating the living create may be exposed to a certain disease or may have exposed someone else to the certain disease if the cloud-based computing system knows the person is already exposed to the certain disease. Further, the function may emit an alert (e.g., visually using a user interface, a light, a display screen; audibly using a speaker; using haptics via a haptic feature) that indicates that the transmission probability exceeds the threshold amount. The function may include presenting a notification that the living creature should be tested and to see a medical professional immediately or to initiate a telemedicine session with a medical professional. Another preventative action may include the cloud-based computing device controlling another electronic device in the physical space to perform a function (e.g., sound an alarm, emit an announcement of the threshold amount of the transmission probability being exceeded in that physical space, or the like). Further, another preventative action may include the cloud-based computing device controlling a user device 12 of a medical professional (e.g., a nurse) that is taking care of the person with the transmission probability exceeding the threshold amount. The cloud-based computing device may cause the user device 12 of the nurse to display a notification indicating the person may have transmitted or been exposed to the certain disease, to administer a test on the person, to take the vital signs of the person, or the like.

These probabilities may be accessed after the interaction in order to engage in contact tracing. For example, if the first living creature is later determined to be infected with an infectious disease (e.g., COVID-19), the probability that the first living creature infected the second living creature could be used in order to determine whether the second living creature should be quarantined or tested. This can be repeated for additional living creatures.

At block 1524, the processing device may overlay the paths on a virtual representation of the physical space. This may be used to help visualize the spread of infection or the extent to which social distancing restrictions are being followed.

At block 1526, the processing device may depict an amount of time spent at a time and location intersection of the paths. This amount of time may be used in visualizing how likely it was that transmission occurred.

At block 1528, the processing device may depict an amount of time spent at a zone of a plurality of zones along one of the paths when an input at the computing device is received that corresponds to the zone. This information, along with the amounts of time spent at each of the zones along other paths may allow visualization of hot spots and aid in changing the arrangement of the physical space to reduce the potential for spread of coronavirus.

Consistent with the above disclosure, the examples of systems and method enumerated in the following clauses are specifically contemplated and are intended as a non-limiting set of examples.

Clause 1. A method for tracking potential disease spread in a physical space, the method comprising:
receiving, at a first time in a time series from a device in the physical space, first data pertaining to a first initiation event of a first path of a first living creature in the physical space;
receiving, at a second time in the time series from one or more smart floor times in the physical space, second data pertaining to a first time and location event caused by the first living creature in the physical space, wherein the first time and location event comprises a first initial location of the first living creature in the physical space; and
correlating, via a processing device, the first initiation event and the first initial location to generate a first starting point comprising a first starting time and first starting location of a first path of the first living creature in the physical space.

Clause 2. The method of any preceding clause, further comprising:
receiving, at a third time in the time series from a device in the physical space, third data pertaining to a second initiation event of a second path of a second living creature in the physical space;
receiving, at a fourth time in the time series from one or more smart floor tiles in the physical space, fourth data pertaining to a second time and location event caused by the second living creature in the physical space, wherein the second time and location event comprises a second initial time and location of the second living creature in the physical space; and
correlating, via a processing device, the second initiation event and the initial location to generate a second starting point comprising a second starting time and second starting location of a second path of the second living creature in the physical space.

Clause 3. The method of any preceding clause, further comprising:
receiving, at a fifth time in the time series from the one or more smart floor tiles in the physical space, fifth data pertaining to one or more first subsequent time and location events caused by the first living creature in the physical space, wherein the one or more first subsequent time and location events comprise one or more first subsequent times and one or more first subsequent locations of the first living creature in the physical space; and
generating the first path comprising the first starting point and the one or more first subsequent locations of the first living creature;
receiving, at a sixth time in the time series from the one or more smart floor tiles in the physical space, sixth data pertaining to one or more second subsequent time and location events caused by the second living creature in the physical space, wherein the one or more second subsequent time and location events comprise one or more second subsequent times and one or more second subsequent locations of the second living creature in the physical space; and
generating the second path comprising the second starting point and the one or more second subsequent locations of the second living creature.

Clause 4. The method of any preceding clause, further comprising:
using the one or more first subsequent times, the one or more first subsequent locations, the one or more second subsequent times, and the one or more second subsequent locations, determine one or more distances between the first living creature and the second living creature;
using the one or more distances, calculate one or more transmission probabilities; and
determine whether at least one of the one or more transmission probabilities exceeding a minimum transmission probability threshold.

Clause 5. The method of any preceding clause, further comprising:
using the first path and the second path, determining a transmission probability between the first living creature and the second living creature.

Clause 6. The method of any preceding clause, further comprising:
overlaying the first path and the second path on a virtual representation of the physical space; and
depicting an amount of time spent at a time and location intersection of the first path and the second path.

Clause 7. The method of any preceding clause, further comprising:
depicting an amount of time spent at a zone of a plurality of zones along one of the first path and the second path when an input at the computing device is received that corresponds to the zone.

Clause 8. The method of any preceding clause, wherein the first time and the second time differ less than a threshold period of time, or the first time and the second time are substantially the same.

Clause 9. The method of any preceding clause, wherein the initial location is generated by one or more detected forces at the one or more smart floor tiles.

Clause 10. The method of any preceding clause, wherein the living creature is a person, and the first data comprises:
a gender of the person,
an age of the person,
a disease risk factor of the person,
whether the person is wearing a face mask,
an identity of the person,
an employment position of the person in an entity,
the entity for which the person works, or
some combination thereof.

Clause 11. The method of any preceding clause, wherein the living creature is a person, the method comprises detecting a body temperature of the person, and the first data comprises the body temperature of the person.

Clause 12. A system comprising:
a memory device storing instructions; and
a processing device communicatively coupled to the memory device, the processing device executes the instructions to:
receive, at a first time in the time series from a device in the physical space, first data pertaining to a first initiation event of a first path of a first living creature in the physical space;
receive, at a second time in the time series from one or more smart floor tiles in the physical space, second data pertaining to a first time and location event caused by the first living creature in the physical space, wherein the first time and location event comprises a first initial location of the first living creature in the physical space; and
correlate, via a processing device, the first initiation event and the first initial location to generate a first starting point comprising a first starting time and first starting location of a first path of the first living creature in the physical space.

Clause 13. The system of any preceding clause, wherein the processing device further executes the instructions to:
receive, at a third time in the time series from a device in the physical space, third data pertaining to a second initiation event of a second path of a second living creature in the physical space;
receive, at a fourth time in the time series from one or more smart floor tiles in the physical space, fourth data pertaining to a second time and location event caused by the second living creature in the physical space, wherein the second time and location event comprises a second initial time and location of the second living creature in the physical space; and
correlate, via a processing device, the second initiation event and the initial location to generate a second starting point comprising a second starting time and second starting location of a second path of the second living creature in the physical space.

Clause 14. The system of any preceding clause, wherein the processing device further executes the instructions to:
receive, at a fifth time in the time series from the one or more smart devices tiles in the physical space, fifth data pertaining to one or more first subsequent time and location events caused by the first living creature in the physical space, wherein the one or more first subsequent time and location events comprise one or more first subsequent times and one or more first subsequent locations of the first living creature in the physical space; and
generate the first path comprising the first starting point and the one or more first subsequent locations of the first living creature;
receive, at a sixth time in the time series from the one or more smart floor tiles in the physical space, sixth data pertaining to one or more second subsequent time and location events caused by the second living creature in the physical space, wherein the one or more second subsequent time and location events comprise one or more second subsequent times and one or more second subsequent locations of the second living creature in the physical space; and
generate the second path comprising the second starting point and the one or more second subsequent locations of the second living creature.

Clause 15. The system of any preceding clause, wherein the processing device further executes the instructions to:
using the first path and the second path, determine a transmission probability between the first living creature and the second living creature.

Clause 16. The system of any preceding clause, wherein the processing device further executes the instructions to:
overlay the first path and the second path on a virtual representation of the physical space; and
depict an amount of time spent at a time and location intersection of the first path and the second path.

Clause 17. The system of any preceding clause, wherein the processing device further executes the instructions to:
depict an amount of time spent at a zone of a plurality of zones along one of the first path and the second path when an input at the computing device is received that corresponds to the zone.

Clause 18. The system of any preceding clause, wherein the living creature is a person, and the first data comprises a detected body temperature of the person.

Clause 19. A tangible, non-transitory computer-readable medium storing instructions that, when executed, cause a processing device to:
receive, at a first time in a time series from a device in the physical space, first data pertaining to a first initiation event of a first path of a first living creature in the physical space;
receive, at a second time in the time series from one or more smart floor times in the physical space, second data pertaining to a first time and location event caused by the first living creature in the physical space, wherein the first time and location event comprises a first initial location of the first living creature in the physical space; and
correlate, via a processing device, the first initiation event and the first initial location to generate a first starting point comprising a first starting time and first starting location of a first path of the first living creature in the physical space.

Clause 20. The tangible, non-transitory computer-readable medium of any preceding clause, wherein the living creature is a person, the instructions cause the processor to cause a device to detect a body temperature of the person, and the first data comprises the body temperature of the person.

The various aspects, embodiments, implementations or features of the described embodiments can be used separately or in any combination. The embodiments disclosed herein are modular in nature and can be used in conjunction with or coupled to other embodiments, including both statically-based and dynamically-based equipment. In addition, the embodiments disclosed herein can employ selected equipment such that they can identify individual users and auto-calibrate threshold multiple-of-body-weight targets, as well as other individualized parameters, for individual users.

The foregoing description, for purposes of explanation, used specific nomenclature to provide a thorough understanding of the described embodiments. However, it should be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It should be apparent to one of ordinary skill in the art that many modifications and variations are possible in view of the above teachings.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

1. A method for tracking potential disease spread in a physical space, the method comprising:
receiving, at a first time in a time series from a device in the physical space, first data pertaining to a first initiation event of a first path of a first living creature in the physical space;
receiving, at a second time in the time series from one or more smart floor times in the physical space, second data pertaining to a first time and location event caused by the first living creature in the physical space, wherein the first time and location event comprises a first initial location of the first living creature in the physical space; and
correlating, via a processing device, the first initiation event and the first initial location to generate a first starting point comprising a first starting time and first starting location of a first path of the first living creature in the physical space.

2. The method of claim 1, further comprising:
receiving, at a third time in the time series from a device in the physical space, third data pertaining to a second initiation event of a second path of a second living creature in the physical space;
receiving, at a fourth time in the time series from one or more smart floor tiles in the physical space, fourth data pertaining to a second time and location event caused by the second living creature in the physical space, wherein the second time and location event comprises a second initial time and location of the second living creature in the physical space; and
correlating, via a processing device, the second initiation event and the initial location to generate a second starting point comprising a second starting time and second starting location of a second path of the second living creature in the physical space.

3. The method of claim 2, further comprising:
receiving, at a fifth time in the time series from the one or more smart floor tiles in the physical space, fifth data pertaining to one or more first subsequent time and location events caused by the first living creature in the physical space, wherein the one or more first subsequent time and location events comprise one or more first subsequent times and one or more first subsequent locations of the first living creature in the physical space; and
generating the first path comprising the first starting point and the one or more first subsequent locations of the first living creature;
receiving, at a sixth time in the time series from the one or more smart floor tiles in the physical space, sixth data pertaining to one or more second subsequent time and location events caused by the second living creature in the physical space, wherein the one or more second subsequent time and location events comprise one or more second subsequent times and one or more second subsequent locations of the second living creature in the physical space; and
generating the second path comprising the second starting point and the one or more second subsequent locations of the second living creature.

4. The method of claim 3, further comprising:
using the one or more first subsequent times, the one or more first subsequent locations, the one or more second subsequent times, and the one or more second subsequent locations, determine one or more distances between the first living creature and the second living creature;
using the one or more distances, calculate one or more transmission probabilities; and
determine whether at least one of the one or more transmission probabilities exceeding a minimum transmission probability threshold.

5. The method of claim 3, further comprising:
using the first path and the second path, determining a transmission probability between the first living creature and the second living creature.

6. The method of claim 3, further comprising:
overlaying the first path and the second path on a virtual representation of the physical space; and
depicting an amount of time spent at a time and location intersection of the first path and the second path.

7. The method of claim 3, further comprising:
depicting an amount of time spent at a zone of a plurality of zones along one of the first path and the second path when an input at the computing device is received that corresponds to the zone.

8. The method of claim 1, wherein the first time and the second time differ less than a threshold period of time, or the first time and the second time are substantially the same.

9. The method of claim 1, wherein the initial location is generated by one or more detected forces at the one or more smart floor tiles.

10. The method of claim 1, wherein the living creature is a person, and the first data comprises:
a gender of the person,
an age of the person,
a disease risk factor of the person,
whether the person is wearing a face mask,
an identity of the person,
an employment position of the person in an entity,
the entity for which the person works, or
some combination thereof.

11. The method of claim 1, wherein the living creature is a person, the method comprises detecting a body temperature of the person, and the first data comprises the body temperature of the person.

12. A system comprising:
a memory device storing instructions; and
a processing device communicatively coupled to the memory device, the processing device executes the instructions to:
receive, at a first time in the time series from a device in the physical space, first data pertaining to a first initiation event of a first path of a first living creature in the physical space;
receive, at a second time in the time series from one or more smart floor tiles in the physical space, second data pertaining to a first time and location event caused by the first living creature in the physical space, wherein the first time and location event comprises a first initial location of the first living creature in the physical space; and
correlate, via a processing device, the first initiation event and the first initial location to generate a first starting point comprising a first starting time and first starting location of a first path of the first living creature in the physical space.

13. The system of claim 12, wherein the processing device further executes the instructions to:
receive, at a third time in the time series from a device in the physical space, third data pertaining to a second initiation event of a second path of a second living creature in the physical space;
receive, at a fourth time in the time series from one or more smart floor tiles in the physical space, fourth data pertaining to a second time and location event caused by the second living creature in the physical space, wherein the second time and location event comprises a second initial time and location of the second living creature in the physical space; and
correlate, via a processing device, the second initiation event and the initial location to generate a second starting point comprising a second starting time and second starting location of a second path of the second living creature in the physical space.

14. The system of claim 13, wherein the processing device further executes the instructions to:
receive, at a fifth time in the time series from the one or more smart devices tiles in the physical space, fifth data pertaining to one or more first subsequent time and location events caused by the first living creature in the physical space, wherein the one or more first subsequent time and location events comprise one or more first subsequent times and one or more first subsequent locations of the first living creature in the physical space; and
generate the first path comprising the first starting point and the one or more first subsequent locations of the first living creature;
receive, at a sixth time in the time series from the one or more smart floor tiles in the physical space, sixth data pertaining to one or more second subsequent time and location events caused by the second living creature in the physical space, wherein the one or more second subsequent time and location events comprise one or more second subsequent times and one or more second subsequent locations of the second living creature in the physical space; and
generate the second path comprising the second starting point and the one or more second subsequent locations of the second living creature.

15. The system of claim 14, wherein the processing device further executes the instructions to:
using the first path and the second path, determine a transmission probability between the first living creature and the second living creature.

16. The system of claim 14, wherein the processing device further executes the instructions to:
overlay the first path and the second path on a virtual representation of the physical space; and
depict an amount of time spent at a time and location intersection of the first path and the second path.

17. The system of claim 14, wherein the processing device further executes the instructions to:
depict an amount of time spent at a zone of a plurality of zones along one of the first path and the second path when an input at the computing device is received that corresponds to the zone.

18. The system of claim 12, wherein the living creature is a person, and the first data comprises a detected body temperature of the person.

19. A tangible, non-transitory computer-readable medium storing instructions that, when executed, cause a processing device to:
receive, at a first time in a time series from a device in the physical space, first data pertaining to a first initiation event of a first path of a first living creature in the physical space;
receive, at a second time in the time series from one or more smart floor times in the physical space, second data pertaining to a first time and location event caused by the first living creature in the physical space, wherein the first time and location event comprises a first initial location of the first living creature in the physical space; and
correlate, via a processing device, the first initiation event and the first initial location to generate a first starting point comprising a first starting time and first starting location of a first path of the first living creature in the physical space.

20. The tangible, non-transitory computer-readable medium of claim 19, wherein the living creature is a person, the instructions cause the processor to cause a device to detect a body temperature of the person, and the first data comprises the body temperature of the person.
